SEQUENCE LISTING (18 sequences)

SEQ ID NO: 1 - protein, 6 aa, Artificial Sequence
  Synthetic peptide
SEKDEL

SEQ ID NO: 2 - protein, 4 aa, Artificial Sequence
  Synthetic Peptide
KDEL

SEQ ID NO: 3 - protein, 20 aa, Artificial Sequence
  core CPV epitope
SDGAVQPDGGQPAVRNERAT

SEQ ID NO: 4 - DNA, 26 bp, Artificial Sequence
  primer
gttcatccttttcactgaattctgcg

SEQ ID NO: 5 - DNA, 26 bp, Artificial Sequence
  primer
cgcagaattcagtgaaaaggatgaac

SEQ ID NO: 6 - protein, 10 aa, Artificial Sequence
  HA epitope
FERFEIFPKE

SEQ ID NO: 7 - DNA, 40 bp, Artificial Sequence
  primer
ggtgatcatcacattcaagagcggtgaaacatttcaagtc

SEQ ID NO: 8 - DNA, 21 bp, Artificial Sequence
  primer
atgataatcatcgcaagaccg

SEQ ID NO: 9 - DNA, 38 bp, Artificial Sequence
  primer
gtgacgaagacaacatgaataaagtaaaatgttatgtt

SEQ ID NO: 10 - DNA, 50 bp, Artificial Sequence
  primer
gtgacgaattctatggatcccctggagcgtagtttttcatactgattgcc

SEQ ID NO: 11 - DNA, 22 bp, Artificial Sequence
  primer
gtgacggatccggcttccaagg

SEQ ID NO: 12 - DNA, 37 bp, Artificial Sequence
  primer
gtgacgaagacaagatcttacttgtacaactcatcca

SEQ ID NO: 13 - DNA, 52 bp, Artificial Sequence
  primer
gatcgacccgggagatctaagtcatcagaccgtctgacttctggagaactgc

SEQ ID NO: 14 - DNA, 35 bp, Artificial Sequence
  primer
tctggtggatccgcagatcactcaacgacctccgg

SEQ ID NO: 15 - DNA, 32 bp, Artificial Sequence
  primer
tctggtctcgagatccccatatttgttccatc

SEQ ID NO: 16 - DNA, 40 bp, Artificial Sequence
  primer
gatcgaggatccaagtcatcaaactaaagaccgcatttcg

SEQ ID NO: 17 - DNA, 28 bp, Artificial Sequence
  primer
gatccatggcaaaaatcactttgtcatc

SEQ ID NO: 18 - DNA, 33 bp, Artificial Sequence
  primer
atcggatccctattaagcggctctagctaattg

(19) United States
(12) Patent Application Publication
(10) Pub. No.: US 2008/0286297 A1
(43) Pub. Date: Nov. 20, 2008

(54) Chimeric Carrier Molecules for the Production of Mucosal Vaccines

(75) Inventors: Dionisius Elisabeth Antonius Florack, Wageningen (NL); Hendrik Jan Bosch, Wageningen (NL)

Correspondence Address: HOFFMANN & BARON, LLP, 6900 JERICHO TURNPIKE, SYOSSET, NY, 11791, US

(73) Assignee: PLANT RESEARCH INTERNATIONAL B.V., Wageningen (NL)

(21) Appl. No.: 10/574,888

(22) PCT Filed: Oct. 8, 2004
(86) PCT No.: PCT/NL2004/000708

§ 371 Date: Feb. 8, 2007

(30) Foreign Application Priority Data
Oct. 9, 2003 (EP) 03078192.6

(51) Int. Cl. A61K 39/00; C07K 14/195; C12P 21/06; A61K 36/00; C12N 5/10

(52) U.S. Cl. 424/195.11; 530/350; 435/69.1; 435/419; 424/93.7

(57) ABSTRACT

The invention relates to a protein complex for the delivery of an antigen to and across mucosal surfaces and the production of said complex in a host cell, such as a plant. Provided is a protein complex comprising at least two, preferably identical, subunits wherein at least one subunit is unaltered and at least one subunit is fused to a first molecule of interest and wherein the protein complex is able to interact with a cell surface receptor via said subunits. Also provided is a method for producing a protein complex according to the invention, comprising a) providing a host cell with a nucleotide sequence encoding an unaltered subunit and a nucleotide sequence encoding a molecule of interest, wherein at least one molecule of interest is fused to a subunit; b) culturing said host cell thereby allowing expression of said nucleotide sequences and allowing for assembly of the protein complex; c) isolating the complex; d) determining the binding of the complex to a cell surface receptor or to a molecule which mimics a cell surface receptor

The invention relates to the development, composition and the production of mucosal (e.g. oral or nasal) vaccines. More specifically, the invention relates to a protein complex for the delivery of an antigen to and across mucosal surfaces and the production of said complex in a host cell, such as a plant.

Oral vaccination is regarded to be an attractive alternative for injected vaccines because an oral vaccine is, generally speaking, easy to apply and relatively cheap and safe. More important, it can induce protection at the mucosal level, i.e. at the site of entrance of many pathogens. Vaccine administration at a mucosal site, for example by oral or nasal delivery, may even be a prerequisite for the production of vaccines against certain pathogens for which no vaccine is currently available (e.g. respiratory syncytial virus and even possibly HIV). In addition, oral vaccination enables mass vaccination via food or drinking water.

However, oral vaccination often appears not to be very effective. The immune response is short-lasting and typically large doses of antigen are required to elicit the desired effect, even when alive micro-organisms are used. This is due in part to inefficient uptake of antigen (Lavell et al. Adv Drug Delivery Rev 1995; 18:5-22). As a result, various strategies for effective delivery of antigens by mucosal routes have been investigated (see for example O'Hagan, J Pharm Pharmacol (1997)49:1-10; Husband, Vaccine (1193) 11:107-112). Antigens which are successfully delivered across the barrier of epithelial cells lining mucosal tracts stimulate underlying inductive sites of the mucosa-associated lymphoid tissue (MALT). Antigen-specific lymphocytes which are sensitised in the MALT migrate through the circulatory system to populate distant mucosal sites. Thus, mucosal immunisation immunisation may provide both local and systemic protection.

One strategy to develop mucosal vaccines has been to employ bacterial enterotoxins such as cholera toxin (CT) from Vibrio cholera and the related E. coli heat-labile enterotoxin (LT), which are highly immunogenic when delivered mucosally and which can act as carrier molecules and adjuvant to potentiate responses to non-related antigens, the latter especially when the holotoxin is used.

The heat-labile enterotoxin of enterotoxigenic Escherichia coli (LT) and related cholera toxin (CT) from Vibrio cholerae are extremely potent immunogens and mucosal adjuvants upon oral administration (for review see Spangler, 1992 and Williams et al., 1999). Both toxins comprise an A subunit and a pentameric ring of identical B subunits. The A subunit is the toxic part of the chimaeric molecule and causes ADP-ribosylation of G₈_{_{α}} activating adenylate cyclase leading to elevation of cyclic AMP levels. This ultimately results in water loss into the gut lumen characterized by watery diarrhoea. The primary function of the strong non-covalently associated complex comprising the pentameric B subunit is in mediating receptor interactions that result in internalisation and uptake of the toxic A subunit. The primary receptor of the ring of B subunits is the monosialoganglioside GM1 [Gal(β1-3)GalNAcβ(1-4)(NeuAc(α2-3))Galβ(1-4)Glc(β1-1)ceramide], a glycosphingolipid found ubiquitously on the cell surface of mammalian cells including small intestine (Holmgren, 1973; Holmgren et al., 1973, 1975). This unique feature makes the B subunit crucial with respect to triggering the key immunomodulatory events associated with adjuvant activity. It also turns the B subunit into a powerful so-called mucosal carrier molecule. A mucosal carrier molecule is a molecule that interacts, e.g. via a receptor, with immuno-active cells located on the surface of mucosae, such as the mucosa of intestinal epithelium of the small intestine.

The B subunits of both LT (LTB) and CT (CTB) have been successfully used as mucosal carrier molecule in translational fusions with diverse antigens to shuttle these across the gut mucosal epithelium by receptor-mediated uptake (e.g. Dertzbaugh and Elson, 1993; Jagusztyn-Krynicka et al., 1993). Presumably, fusion of an antigen to the carrier molecule enhances the amount of antigen delivered to the MALT inductive sites and the subsequent stimulation of antigen-specific B- and T-lymphocytes. Antigen can also be chemically coupled to LTB (O'Dowd et al. Vaccine (1999) 17:1442-1453; Green et al. Vaccine (1996)14:949-958). In addition, both LTB and CTB also improved immune responses upon oral uptake when co-administered along with antigens (Bowen et al., 1994; Wilson et al, 1993).

Rigano et al. (2003, Vaccine 21:809-811) discloses the generation of an LTB-ESAT6 antigen fusion complex in transgenic Arabidopsis. Expression of the fusion protein results in a homo-pentameric (LTB-ESAT6)₅ complex. ESAT6 is a very small antigen which, accordingly, does not interfere with LTB pentamer formation when fused to LTB. Kim et al. (2003; Plant Cell Reports Vol. 21, no. 9, pp. 884-890) reports the expression of a CTB-NSP175 fusion protein in potato, which gives rise to homopentameric complexes. Whereas genetic fusion of antigens or epitopes to LTB or CTB has been successful in some cases, it appeared that translational fusion of heterologous epitopes to the B subunits can interfere with the structure, secretion, GM1-binding and immunogenicity of the LTB or CTB fusion proteins, as reported for example by Sandkvist et al (J Bacteriol 1987 169:4570), Schodel et al. (Gene 1991;99:255) and Dertzbaugh et al. (Infect Immun 1993; 61:384).

Apparently, there are limitations to the size and type of antigen which can be attached to LTB or CTB such that pentamerization and GM1 binding are retained. For the development of an LTB- or CTB-based vaccine this limitation is especially relevant since most functional vaccines are composed of large structural proteins. In addition, many protective antigens (viral, bacterial and others) are composed of multimeric complexes, either homomultimeric or heteromultimeric, and only induce a protective immune response when delivered as such. For example, a classical swine fever (CSFV) E2 glycoprotein-based vaccine contains a CSFV-E2 homodimer. The multimeric nature of various protective antigens greatly reduces the use of LTB and/or CTB as carrier molecule using conventional genetic fusions of these types of antigens, since in this way a complex of five identical fusion proteins is formed.

For commercialisation and market introduction, large-scale production of the fusion protein is required and the product needs to be safe and nearly pure. Production of such vaccines based on fusion-proteins of LTB and CTB and antigens, in bacteria and yeast requires large-scale fermentation technology and stringent purification protocols to obtain sufficient amounts of recombinant protein for oral delivery. Transgenic plants and especially edible plant parts, are safe expression systems for vaccines for oral delivery (for review see Langridge, 2000; Mason and Arntzen, 1995; Sala, 2003). Recently, LTB and CTB were successfully produced in diverse plant species, including tobacco, potato and corn (Arakawa et al., 1997; Haq et al., 1995; Hein et al., 1995; Lauterslager et al., 2001; Streatfield et al., 2001). Surprisingly, also plants appeared able to produce pentamers of the B subunits similar to E. coli and V. cholerae.

Several groups have reported that oral immunisation of mice by means of feeding agave, potato tubers or corn accumulating either LTB or CTB resulted in both serum IgG and secretory sIgA responses (Arakawa et al., 1998; Haq et al., 1995; Lauterslager et al., 2001; Mason et al., 1998; Streatfield et al., 2001). WO-A-991825 describes the oral immunization of mice with CTB subunits fused to a SEKDEL sequence produced in plants.

A pre-clinical trial with human volunteers fed 50 to 100 grams amounts of transgenic tubers accumulating LTB also resulted in specific serum IgG and secretory sIgA responses Tacket et al., 1998). Accordingly, transgenic host cell systems are in essence advantageously used for the large-scale) production of mucosal carrier molecules such as a LTB- or CTB-fusion protein. However, it has been shown that the expression level of a fusion protein significantly decreases with increasing size and complexity of the fusion protein. For example, expression of the gene construct encoding LTB fused to the CSFV-E2 glycoprotein (approximately 35 kDa and protective as a dimer of appr. 70 kDa) in potato tubers was more than 100 times lower than that of the gene encoding the LTB subunit of ˜15 kDa (Lauterslager, 2002; PhD thesis University of Utrecht). Possibly, the low expression levels of large LTB- or CTB-fusion proteins fundamentally reflect instability of the large pentameric protein complex because it is often observed that monomers are more prone to (enzymatic) degradation than assembled multimeric structures. This is consistent with Arakawa et al. (1999; Transgenics, Harwood Academic Publishers, Basel, Vol. 3, no. 1, pp. 51-60). who reported the accumulation of the CTB-GAD fusion protein in potato. GAD (human glutamate decarboxylase) is a 65 kDa autoantigen. Quantitation of CTB-GAD revealed that the expression level was only 0.001% of total soluble protein, clearly indicating the problems that can be encountered when trying to accumulate significant levels of such high molecular weight complexes. However, the low amount of active homopentameric complex appeared to be immunogenic when mice were fed for a prolonged time period and with high amounts of fresh transgenic potato material.

Thus, it is an object of the present invention to provide functional carrier complexes that allow for the delivery of relatively large antigens to a site of interest without compromising the expression level of the complex, e.g. in a recombinant host cell.

The invention now provides the insight that the assembly, functionality and stability of a multisubunit carrier molecule is enhanced, if not all but only some of the subunits are fused to an antigen or other molecule of interest. Provided is a protein complex comprising at least two, preferably identical, subunits wherein at least one subunit is unaltered and at least one subunit is fused to a first molecule of interest and wherein the protein complex is able to bind to a cell surface receptor.

In a preferred embodiment, said protein complex is a carrier molecule that can be used to carry or transport a molecule of interest. For example, a protein complex of the invention is a carrier complex or carrier molecule for the delivery of a molecule of interest to a site of interest, e.g. to and/or across a mucosal surfaces. According to the invention, a molecule of interest can comprise a variety of different classes of proteins or polypeptides or stretches thereof, that one may wish to deliver at, distribute within, transport to or retain at a certain site in an organism using the carrier properties of the complex. It especially refers to the moiety fused or added to a subunit that, when one would try to attach it to all subunits of the carrier, would interfere with the formation of a stable and functional multimeric structure of a carrier molecule e.g. by sterical hindrance. For instance, the size of said molecule of interest fused to a subunit is a quarter, a third, half, once, twice, three or even four times the size of the subunit alone.

The term “unaltered” as used herein refers to a subunit or monomer that is not fused to a molecule of interest. It is however not limited to the native subunit or monomer; for example it is a recombinant protein wherein one or more amino acids are removed from, replaced in or added to the native subunit. This may be done to modulate the stability and/or the production process, e.g. expression or secretion, of the recombinant protein in a (eukaryotic) host cell. For instance, an unaltered subunit may comprises a subunit provided with a signal peptide or a (SE)KDEL sequence at the C-terminus for retention in the endoplasmic reticulum (ER). The term “unaltered subunit” essentially refers to a native subunit or a slightly modified version thereof wherein the modification does not interfere with multimerization.

WO-A-9612801 discloses the coordinate expression of an LTA subunit and an LTB subunit fused to a SEKDEL sequence which upon expression together form the CT holotoxin protein complex. Such a protein complex is distinct from a protein complex according to the invention, since it does not comprise an subunit fused to a molecule of interest according to the present invention. The SEKDEL hexapeptide is not regarded as a molecule of interest. Rather, as noted above, the LTB-SEKDEL subunit is regarded as an “unaltered” subunit. Consequently, according to the terminology of the present invention the complex of WO-A-9612801 is distinct from a protein complex provided herein as it comprises unaltered subunits only.

U.S. Pat No 6,103,243 describes oral vaccines and method to improve the uptake of immunogens, e.g. by using mucosal carrier complexes such as LTB. LTB subunits are provided with antigens of interest, either by chemical linkage or by genetic fusions. This results in homopentameric protein complexes in which all subunits are fused to one type of molecule of interest. Thus, unlike the present invention, a complex of U.S. Pat. No. 6,103,243 does not comprise at least one unaltered subunit and at least one subunit fused to a molecule of interest.

In a preferred embodiment of the present invention, a protein complex comprises at least two identical subunits, e.g. LTB subunits, of which at least one subunit is altered an at least one subunit is fused to a molecule of interest. In one aspect of the invention, a protein complex comprises at least two, preferably identical, subunits characterised in that at least one subunit is unaltered and at least one subunit is fused to a first molecule of interest and wherein said first molecule of interest can associate with a second molecule of interest to form a multimer of interest, and wherein the protein complex is able to interact with a cell surface receptor via said subunits. Preferably, said first molecule associates or interacts with said second molecule via an intermolecular covalent bond, for instance via one or more disulfide bridge(s). A multimer of interest according to the invention is for example a multimeric protective antigen such as a homodimeric or a heterodimeric antigen. Known multimeric protective antigens include the CSFV-E2 homodimer, the trimeric glycoprotein G of viral haemorrhagic septicaemia virus (VHSV-G) (Lorenzen, N., Lorenzen, E., Einer-Jensen, K., Heppell, J., Wu, T., Davis, H. (1998). Protective immunity to VHS in rainbow trout (Oncorhynchus mykiss, Walbaum) following DNA vaccination. Fish & Shellfish Immunology 8: 261-270; Lorenzen, N., Olesen, N. J. (1997) Immunisation with viral antigens: viral haemorrhagic septicaemia. In: Fish Vaccinology. Gudding, R., Lillehaug, A., Midlyng, P. J., Brown, F. (eds). Dev. Biol. Stand. Basel, Karger, vol. 90, p 201-209) and trimeric glycoprotein G of Rabies virus (RV G) and vesicular stomatitis virus (VSV G); the homotetrameric phosphoprotein P of Sendai virus (SeV P).

According to the invention, a second molecule of interest may be fused to a subunit. For example, in one embodiment of the invention a protein complex comprises three subunits, two of which are fused to an identical (e.g. CSFV-E2) monomer, and one unaltered subunit. It is desired that the subunits are in dose proximity of each other such that the fused monomers can interact and are capable of forming a homomultimer, for instance an antigenic (e.g. CSFV-E2) homodimer. The presence of an unaltered subunit enhances the stability of the multisubunit structure of the protein complex and ensures that the conformation of the complex is retained such that it can interact via the subunits with its receptor. Obviously, it is also possible that a protein complex of at least three subunits comprises two or more subunits that are fused to different monomers and at least one unaltered monomer, such that the fused monomers can form a heteromultimer.

Of course, a second molecule of interest does not need to be fused to a subunit in order to interact with a first molecule of interest and form a multimer of interest. Moreover, according to the present invention, it may sometimes be advantageous e.g. with respect to steric hindrance, to design a protein complex comprising a multimer of interest wherein said multimer of interest is composed out of multiple molecules of interest that are not all fused to a subunit. For example, the degree of interaction between molecules of interest which are all fused to a subunit is to a certain extent determined by the relative orientation of the individual subunits they are fused to. Accordingly, molecules that are normally (i.e. in their native, non-fused conformation) capable of interacting with each other may become spatially restricted when fused to a subunit resulting in a decrease or even complete loss of interaction between the molecules. Thus, according to the present invention a protein complex may comprise a multimer of interest composed of multiple molecules of interest capable of forming a multimeric structure, for instance through disulfide bridges. Advantageously, a protein complex of the invention comprising at least one unaltered subunit and at least one subunit fused to an antigen typically displays improved folding of one or more (multimeric) antigens into an antigenic moiety. As said before, one, some, or all molecules of interest are fused to a subunit. Based on the quaternary structure and symmetry of the subunits and of multimer of interest, a person skilled in the art will be able to select the optimal number of fused versus non-fused molecules in order to obtain a protein complex comprising a multimer of interest with an optimal configuration, e.g. optimal antigenic properties.

In one embodiment, a protein complex is able to bind to a cell surface receptor that is present on intestinal epithelial, for example to a ganglioside molecule like GM1. Such a protein complex comprising a molecule of interest is advantageously used as a mucosal carrier molecule.

In a preferred embodiment, a protein complex of the invention is essentially based on the heat labile enterotoxin (LT) of E. coli or on the cholera toxin (CT) of Vibrio cholerae, preferably on the B subunits thereof. A complex of the invention is for instance a ring structure composed of five B subunits of LT or CT, wherein at least one subunit is unaltered and at least one subunit is fused to a molecule of interest. Such a chimeric or hetero-pentameric ring structure of the invention comprises one, two, three or four unaltered LTB- or CTB-monomers and four, three, two or one L/CTB-fusion protein, respectively. In a preferred embodiment, a molecule of interest is fused to a subunit such that it is located at the opposite side of the molecule from the receptor-binding pocket, or at least not interfering with receptor binding properties of said subunit. For example, for a complex of the invention that is based on the pentameric LTB- or CTB-structure, this means that a molecule of interest is preferably fused to the C-terminus of the B subunit (Sixma et al., 1991 Nature 351; 371-377). Following this strategy of the invention, chimeric pentamers are obtained which have retained their conformational integrity. Since LTB subunits and CTB subunits are homologous, they can be used to form a protein complex composed of a chimeric LTB/CTB-pentamer wherein at least one subunit (be it LTB or CTB) is fused to a molecule of interest and wherein at least one subunit is unaltered LTB- and/or CTB-based chimeric protein complexes of the invention show improved binding to the GM1 receptor and increased expression levels when compared to a homopentameric structure wherein all five subunits are fused to a molecule of interest.

In another embodiment, a protein complex of the invention is based on horseradish peroxidase (HRP). A complex of the invention is for instance a structure or composition comprised of six subunits of HRP wherein at least one subunit is unaltered and at least one subunit is fused to a molecule of interest. Such a chimeric or hetero-hexameric structure of the invention comprises one, two, three, four or five unaltered HRP-subunits (monomers) and five, four, three, two or one HRP-fusion proteins, respectively.

A complex according to the invention allows for optimal folding of and intramolecular interaction (e.g. via a disulphide bridge) within a multimeric antigen and thus has optimal immunogenic properties.

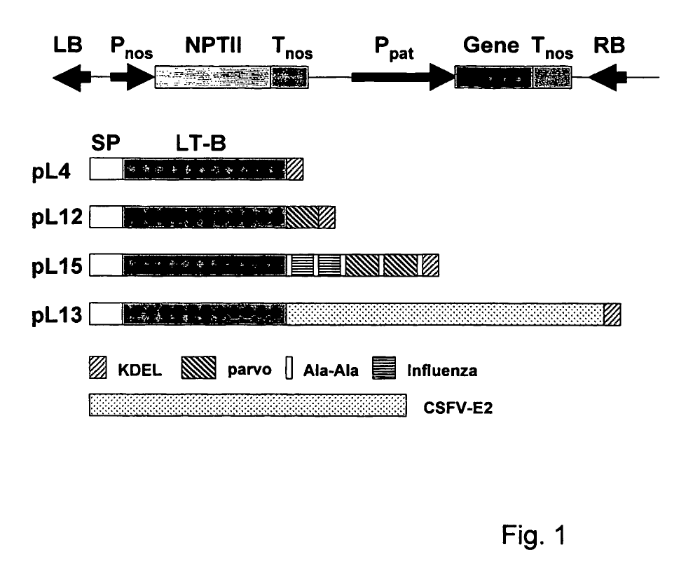

In one embodiment, a molecule of interest is capable of effecting or influencing the immune system of an organism, preferably a mammal, more preferably a human. In a preferred embodiment, a molecule of interest is an antigen. An antigen is any substance that stimulates the immune system. Antigens are often foreign microorganisms such as bacteria or viruses that invade the body or components of said microorganism such as proteins or protein fragments. A molecule of interest is preferably selected from the group consisting of a bacterial antigen, a viral antigen, a protozoal antigen, a nematode antigen and a fungal antigen. The invention provides a protein complex for the delivery of various antigens, for instance T- and/or B-cell epitopes such as the linear B cell epitope CPV (canine parvo virus epitope) and the T-cell specific epitope HA (influenza virus hemagglutinin epitope), and a composition comprising such a protein complex. A complex of the invention may also be used for the delivery of large antigens like for instance a viral (glyco)protein such as the E2 protein of CSFV (classical swine fever virus). One subunit fusion protein may also be fused to multiple molecules of interest. For example, a protein complex is provided comprising at least one unaltered subunit and at least one subunit translationally fused to two HA epitopes and two parvo epitopes (see FIG. 1). In one embodiment of the invention, a protein complex (e.g. an LTB-based complex) comprises at least one subunit which is unaltered and at least one, preferably two, subunits which are fused to the CSFV E2 glycoprotein. In another embodiment, a protein complex of the invention comprises at least one unaltered subunit, at least one subunit which is fused to the parvo epitope and at least one subunit which is fused to multiple molecules of interest, the latter comprising a subunit fused to two HA epitopes and two parvo epitopes.

In a further embodiment, a protein complex of the invention comprises at least one subunit which is unaltered and at least one subunit which is fused to an immunomodulatory molecule (or a part thereof), for example a cytokine or a heat-shockprotein (HSP). A cytokine is the general term for a large group of molecules involved in signalling between cells during an immune response. Cytokines are proteins or polypeptides, some with sugar molecules attached (glycoproteins). Different groups of cytokines can be distinguished: the interferons (IFN alpha, beta, gamma); the interleukins (IL-1 to IL-15); the colony stimulating factors (CSFs) and other cytokines such as tumour necrosis factor (TNF) alpha and beta or transforming growth factor (TGF) beta. HSPs have remarkable immunomodulatory properties which derive from their interaction with macrophage and dendritic cells through a receptor, identified as CD91. For example, HSP70-2 is an important immunomodulatory protein induced in response to inflammatory stimuli. HSPs of interest to include in a protein complex according to the invention comprise HSP-60, HSP-70, HSP-90 and Gp-96. Depending on the type of the desired immunological response (e.g. Th1 versus Th2, antibody response, anti-inflammatory response), one or more (different) immunomodulatory protein(s) or part(s) thereof may be fused to a subunit of a protein complex of the invention. For example, a multicomponent vaccine of the invention comprises a protein complex comprising an antigen and an immunomodulatory molecule, preferably a cytokine, which directs an antibody response (T, B cell). On the other hand, for the treatment or prevention of for example an autoimmune disease (e.g. diabetes, multiple sclerosis) it may be advantageous to use a multicomponent vaccine or a vaccine comprising an auto-antigen and an immunomodulatory protein which directs tolerance.

Other molecules of interest comprise those molecules which, when being part of a complex of the invention, can be used as “reporter” molecule to report the location of a carrier complex within a body. This is among other helpful to monitor the in vivo binding of a complex to a receptor molecule and the (receptor-mediated) transport of the complex across mucosal epithelium. A reporter molecule of interest is for instance an enzyme (chloramphenicol transacetylase (CAT), neomycin phosphotransferase (neo), beta-glucuronidase (GUS) or firefly luciferase; etc.) or a fluorescent protein such as Green Fluorescent Protein (GFP) or a spectral variant thereof.

In one aspect of the invention, a protein complex comprises at least three subunits wherein at least two subunits are provided with a molecule of interest. Said at least two subunits can be translationally fused to the same molecule of interest or to a different molecule of interest. A protein complex of the invention with least two different molecules of interest is advantageously used as a carrier molecule for at least two different antigens, e.g. for the production of a multicomponent vaccine. Other combinations of different types of molecules of interest, for instance one or more antigens with one or more immunomodulatory (either stimulatory or inhibitory) proteins are of course also possible.

A cholera toxin-based multicomponent vaccine was described by Yu and Langridge (Nature, 2001, vol. 19:548), who studied the expression of a cholera toxin B subunit fused to a 22-amino acid immunodominant epitope of the murine rotavirus enterotoxin NSP4, and the ETEC fimbrial colonization factor CFA/I fused to the CTA2 subunit. Unlike in a complex of the invention, all of the subunits (both B and A2) of the reported cholera toxin complex were fused to an antigen. The fact that in this specific case no problems were encountered with CTB expression nor with the formation of a functional CTB/A2 complex was probably related to the very small size (approximately 5 kDa) of the NSP4 epitope fused to the B subunit. As said, problems with functional pentamer formation are largely due to steric hindrance and such a small epitope fused to a subunit is unlikely to interfere with pentamer formation. According to the invention, it is now also possible to produce (multicomponent) vaccines, wherein the antigens are considerably larger than 5 kDa. For example, a protein complex is provided comprising at least one (CT/LT) B subunit fused to molecule of interest, at least one unaltered B subunit and an A2 subunit fused to a different molecule of interest. Preferably, said molecule fused to the B subunit is larger than 7 kDa, more preferred larger than 10 kDa, even more preferred larger than 15 or even 25 kDa. However, in another embodiment of the invention, an (LT/CT). B subunit and an (LT/CT) A2 subunit of the same carrier complex are fused to identical antigens wherein said antigen is part of a multimeric (dimer, trimer, tetramer) protein complex with immunoprotective properties.

The invention further provides a method for producing a protein complex according to the invention, said method comprising: a) providing a host cell with a first nucleotide sequence encoding an unaltered subunit and a second nucleotide sequence encoding a subunit fused to a molecule of interest; b) culturing said host cell thereby allowing expression of said first and second nucleotide sequences and allowing for assembly of the protein complex; c) isolating the complex; and d) determining the binding of the complex to a cell surface receptor.

The term “host cell” refers to any cell capable of replicating and/or transcribing and/or translating a heterologous gene. In one embodiment, a host cell is a plant cell, a phage or a bacterium. In a preferred embodiment, a host cell of the invention is an edible host cell, which does not cause any harmful effects when consumed. Preferred examples are potato, tomato, tobacco, maize and Lactobacillus. Thus, a host cell refers to any eukaryotic or prokaryotic cell (e.g. bacterial cells such as Escherichia coli, yeast cells such as Pichia pastoris, mammalian cells such as Chinese Hamster Ovary cells, avian cells, amphibian cells, plant cells, fish cells, fungal cells such as Agaricus bisporis, and insect cells such as Spodoptera frugiperda), whether located in vitro or in vivo. For example, host cells may be located in a transgenic plant.

As described above, in a specific embodiment, a plant can be used in the method of the present invention. Said plant host maybe a monocot, such as Zea mays, Triticum aestivum, Oryza sativa or Lemna spp., or a dicot, such as plants related to the genus Nicotiana, such as N.tabacum, Lycopersicon, such as tomato, the family Leguminosae, including the genus Medicago, or mosses such as Physcomitrella patens. In a preferred embodiment said host plant is Solanum tuberosum.

According to the invention, a host cell is provided with at least two different nucleic acid sequences: one encoding an unaltered subunit (not fused to an antigen of interest) and one encoding a subunit translationally fused to a molecule of interest. For example, a (plant) host cell is transfected with a first nucleotide construct encoding an unaltered LTB subunit and a second construct encoding an LTB-subunit fused to an antigen of interest. A nucleotide sequence encoding a subunit fused to a molecule of interest may also comprise a linker or hinge region in between the subunit and the molecule of interest to increase the flexibility of the resulting fusion protein. Following transfection and culturing under suitable conditions, said cell will express the two polypeptides to assemble a functional LTB-based chimeric protein complex “loaded” with antigen. To produce a protein complex of the invention with at least two different molecules of interest, a host cell is of course provided with at least three different nucleotide sequences. These different nucleic acid sequences can be introduced into a host cell by co-transformation of said host cell with different vectors (e.g. using T-DNA), each carrying a different nucleotide sequence. Alternatively, two or more different gene constructs can be introduced in one host cell by crossing or by using two or more expression cassettes on one binary vector. Furthermore, an established host cell line already comprising one (or more) of the components of a protein complex according to the invention can be provided with an additional nucleic acid sequence using re-transformation. In yet an alternative embodiment, a host cell expressing at least two different nucleic acid sequences is obtained by the transient (virus-mediated) expression of one sequence in a host cell which stably expresses another sequence.

An advantage of using different vectors can be sought in the fact that it allows for providing a host cell with different nucleic acid constructs in varying ratios. Herewith, it is possible to titrate the amount of unaltered, “free” subunit relative to the amount of fused subunit that is expressed by a host cell and to optimize the composition of the resulting multimeric protein complex. For instance, if a complex is desired which contains predominantly fused subunits, a host cell is co-transfected or co-transformed with construct A (unaltered subunit) and construct B (fused subunit) wherein construct B is in excess of construct A (e.g. A:B=1:3, or 1:5 or even 1:10). In contrast, excess of construct A over construct B is preferably used to increase the changes of an assembled protein complex comprising relatively few fused subunits. The latter is of course prefer red if one wants to minimize negative steric effects or interference of the fused subunit with the assembly of a functional protein complex of the invention.

In yet another embodiment, a host cell is transformed with a nucleic acid construct encoding a subunit fused to a molecule of interest, e.g. LTB-CSFV E2, wherein said construct comprises a proteolytic cleavage site in between the nucleic acid sequence encoding the subunit and the nucleic acid sequence encoding the molecule of interest. Upon the partial in vivo cleavage of such a fusion protein by a protease (expressed endogenous of heterologous in the host cell), the host cell will contain both unaltered subunits as well as subunits fused to a molecule of interest which can form a chimeric protein complex according to the invention.

Various procedures known in the art can be used to provide a host cell with a recombinant or isolated nucleic acid (DNA or RNA). These include transformation, transfection (e.g. using calcium phosphate precipitation or a cationic liposome reagent), electroporation, particle bombardment and Agrobacterium-mediated T-DNA transfer. Depending on the type of host cell, a person skilled in the art will recognize which procedure to choose.

Following providing a host cell with said foreign nucleotides, the host cell is cultured to allow (co-)expression of said first and second nucleotide sequences and assembly of the resulting polypeptides into a protein complex of the invention. In some cases, especially when transformation or transfection procedures are relatively inefficient, it is advantageous to select those host cells which have truly received said nucleotides and to only culture those selected host cells. Host cell selection following transformation or transfection (or other procedures to provide a host cell with an isolated nucleic acid) can be performed according to standard methods. For example, most common vectors used to deliver a DNA sequence of interest to a host also contain a nucleic acid sequence encoding a protein (such as an enzyme) which, upon efficient delivery to and expression by the host cell, provides said host cell with resistance to a selection agent. A frequently used selection agent is an antibiotic, such as neomycin, kanamycin, ampicillin, carbenicillin, etc.

In a method of the invention, a (selected) host cell provided with at least two different nucleic acids (be it using a binary vector or different vectors) will express at least two different polypeptides, e.g. an unaltered (non-fused) subunit X and a fused subunit Y.

In another embodiment of the invention, a first host cell, e.g. a microbial host cell, provides an unaltered subunit X and a second host cell provides a fused subunit Y comprising a molecule of interest, for instance an antigen. Optionally, a third host cell capable of producing fused subunit Z comprising a second molecule of interest, e.g. a second antigen or an immunomodulatory molecule such as a cytokine. Following the isolation of the separately produced X and Y (and optionally Z) subunits, they can be contacted with each other under conditions that are favourable for the formation of a protein complex comprising at least one X subunit and at least one Y subunit. This method of in vitro reconstituting a chimeric protein complex of the invention allows for titrating the number of unaltered versus fused subunits within one complex by contacting them in a certain ratio with each other. In contrast to using a single host cell producing both unaltered and fused subunits, the make-up of a chimeric protein complex according to the above-mentioned reconstitution method is not dependent on the relative expression levels of the subunits. Rather, the ratio between unaltered and fused subunits that are contacted with each other can be controlled (see also Example 9). Herewith, the invention provides a method for producing a protein complex according to the invention, comprising: a) providing a first host cell with a nucleotide sequence encoding an unaltered subunit and a second host cell a nucleotide sequence encoding a molecule of interest, wherein at least one molecule of interest is fused to a subunit; b) culturing said host cells thereby allowing expression of said nucleotide sequences; c) isolating the proteins encoded by said nucleotides; d) contacting the isolated protein under conditions allowing for assembly of the protein complex; e) isolating the complex; f) determining the binding of the complex to a cell surface receptor or to a molecule which mimics a cell surface receptor. As said, in step d) the proteins can be mixed in a specific ratio to favour the formation of a protein complex with ascertain subunit composition.

Assembly of said at least two polypeptides into a multimeric complex can result in various complexes, each with a different subunit composition. In theory, two types of complexes can be formed: homomeric complexes comprising only unaltered X subunits or only fused Y subunits, and heteromeric, or chimeric, complexes comprising a mixture of X and Y subunits. Depending on the number of X and Y subunits present in a complex, various types of chimeric complexes are possible. For example, a trimeric complex may comprise two X subunits and one Y subunit or vice versa; a tetrameric complex may comprise three, two or only one X subunit and one, two or three Y subunits, respectively; and so on. As will be understood, a protein complex of the invention relates to the latter (chimeric) protein complexes; a homomeric X complex lacks a molecule of interest and a homomeric Y complex, if assembled at all, is probably not capable of binding to a receptor because of the presence of steric hindrance by the large number of (bulky) fused molecules of interest. Furthermore, a homomeric protein complex wherein all subunits are fused to an antigen, may not be a useful carrier molecule for multimeric antigens because of a sub-optimal orientation/conformation of the individual antigen monomers with respect to immunogenic properties. Therefore, in one embodiment of the invention a chimeric protein complex as provided herein is isolated from a mixture of chimeric and homomeric complexes. However, it is to be understood that chimeric protein complexes do not need always need to be separated from homomeric complexes. In one embodiment, a composition comprising chimeric as well as homomeric complexes is suitably used as a vaccine. Preferably however, the chimeric complexes are more abundant than the homomeric complexes. For instance, chimeric protein complexes make up at least 50% of the total number of protein complexes, preferably at least 60% more preferably at least 70%.

Chimeric and homomeric protein complexes can be separated from each other based on their size. Because a fused subunit is by definition larger than an unaltered subunit, complexes with different subunit compositions will have different sizes. This difference allows for the isolation of the desired chimeric complexes from a mixture of homomeric and chimeric protein complexes according to the size of the complexes. In a preferred embodiment, a protein complex of the invention is isolated using gel filtration chromatography. Gel filtration chromatography (also known as size-exclusion chromatography or molecular sieve chromatography) can be used to separate proteins according to their size. Standard information regarding protein chromatography can be obtained from handbooks used in the field, such as “Protein Purification: Principles and Practice” by R K Scopes (Springer-Verlag 3rd edition, January 1994; ISBN 0387940723).

Briefly, during gel filtration, a (mixture of) proteins in solution is passed through a column that is packed with semipermeable porous resin. The semipermeable resin has a range of pore sizes that determines the size of proteins that can be separated with the column. This is called the fractionation range or exclusion range of the resin. Proteins larger than the exclusion range of the resin are unable to enter the pores and pass quickly through the column in the spaces between the resin. This is known as the void volume of the column. Small proteins and other low molecular weight substances that are below the exclusion range of the resin enter all the pores in the resin and their movement through the column is slowed because they must pass through the entire volume of the column. Proteins of a size that falls within the exclusion range of the column will enter only a portion of the pores. The movement of these proteins will be slowed according to their size; smaller proteins will move through the column more slowly because they must pass through a larger volume. To separate a protein sample by gel filtration chromatography, the column must first be equilibrated with the desired buffer. This is accomplished by simply passing several column volumes of the buffer through the column. Equilibration is an important step because the equilibration buffer is the buffer in which the protein sample will elute. Next, the sample is loaded onto the column and allowed to enter the resin. Then more of the equilibration buffer is passed through the column to separate the sample and elute it from the column. Fractions are collected as the sample elutes from the column. Larger proteins elute in the early fractions and smaller proteins elute in subsequent fractions.

Gel filtration should ideally be done at cold temperatures because in addition to reducing degradation of the protein complex it also helps reduce diffusion of the sample during the run, which improves resolution. Separation of proteins is enhanced by using a longer column but the longer running time can increase degradation of the protein.

The choice of a chromatography medium is an important consideration in gel filtration chromatography. Some common gel filtration chromatography media are: Sephadex G-50 (suitable for fractionation of proteins in the range of 1-30 kD); Sephadex G-75; Sephadex G-100 (4-150 kD); Sephadex G-200 (5-600 kD); Bio-Gel P-10 (1.5-20 kD); Bio-Gel P-30 (24-40 kD); Bio-Gel P-100 (5-100 kD) and Bio-Gel P-300 (60-400 kD). Sephadex is a trademark of Pharmacia. Bio-Gel is a trademark of Bio-Rad. The best separation occurs for molecules eluting at about 0.6 column volume, but the peaks typically get broader the later they come off. In one embodiment, a wide fractionation range material is used to give an initial cut, eliminating much larger and much smaller proteins, and subsequently a narrower range material is used for best separation. A gel filtration column can be calibrated using standards (proteins) of known molecular weights. A calibration curve can be constructed showing the retention times as a function of the log MW (logarithm of molecular weight). In a method of the invention, homomeric protein complexes are advantageously used to indicate the range wherein the heteromeric complex will elute from the column. This will be explained further in the following example, which elaborates on one of the examples mentioned above. A host cell (referred to as “XY”) is provided which expresses subunits X and Y, both subunits being capable of forming a tetramer with itself and/or each other. Furthermore, a host cell “X” only expressing subunit X (an unaltered subunit) and a host cell “Y” only expressing subunit Y (a subunit fused to a molecule of interest) are provided. A suitable gel filtration medium is selected according to the size of the predicted size of the homomeric X and Y complexes (four times the size of subunits X and Y, respectively). By definition, chimeric protein complexes of the invention will have a size larger than the X homomer and smaller than the Y homomer. Accordingly, the elution volume of all chimeric protein complexes (XYYY; XXYY; and XXXY) will be larger than that of the Y homotetramer (being the largest complex) but smaller than that of the X homotetramer (being the smallest complex). Application of a protein sample of host cell “Y” containing only Y homotetramers and a protein sample of host cell “X” containing only X homotetramers to a gel filtration column can easily reveal the “boundaries” of the elution volume of chimeric proteins produced by host cell “XY”. For example, column fractions are collected and analysed for the presence of X or Y subunits by SDS-PAGE followed by Western blotting using specific reagents e.g. antibodies. If desired, conditions (column size, column diameter, flow rate etc.) can be adjusted to optimize the resolution of X tetramers from Y tetramers. Of course, the larger the size difference between X and Y (i.e. the larger the molecule of interest), the more easy it will be to resolve different tetrameric complexes from each other. However, the invention specifically relates to solving problems caused by large size differences (e.g. complexes with large antigens). Therefore, separation of complexes according to the invention should not cause major problems. Once the elution volume of chimeric complexes is determined, a protein sample of host cell “XY” can be applied to the column and eluted from the column under the same conditions as were used for the calibration with homotetramers. Column fractions are collected and analysed for the presence of X and/or Y subunits as described above SDS-PAGE of non-boiled protein fraction can be performed to analyze the size of the intact protein complex. Fractions containing a chimeric complex of the invention while being devoid of a homomeric complex are saved for further use. Fractions can either be pooled together to yield a mixture of protein complexes of the invention Fractions can also be kept apart e.g. to yield the complexes XYYY, XXYY and XXXY as separately isolated complexes.

Conventional gel filtration chromatography can be a time consuming process. The procedure can be significantly speed up is the particle size of the chromatography medium or resin is reduced and the column is made smaller. This requires special equipment using higher pressure to get the liquid to flow through the column. In a preferred embodiment, a protein complex of the invention is isolated using high pressure liquid chromatography (HPLC) or fast performance liquid chromatography (FPLC).

In a method of the invention, an isolated protein complex is further characterized to determine its capacity to bind to a cell surface receptor or to a molecule mimicking the receptor binding moiety, e.g. D-galactose mimicking the GM1 receptor can be used for characterizing (or purifying) an LTB- or CTB-based protein complex of the invention. In a preferred embodiment, binding comprises permanent binding since this can be detected more easily than transient binding. Isolated or purified receptors can be used but also cells expressing a receptor, or membrane material derived of these cells, may be used. A protein complex of the invention is contacted with a receptor under conditions that are suitable for binding of the complex to its receptor. Conditions that may influence receptor binding include ionic strength (pH; salts) and temperature. Thereafter, the amount of bound complex is determined. In a preferred embodiment, binding of an isolated protein complex of the invention to a cell surface receptor is performed by an enzyme-linked immunosorbent assay (ELISA) or a procedure essentially based thereon. The basic principle of an ELISA is to use an enzyme to detect the binding of an antigen (Ag) to an antibody (Ab). The enzyme converts a colorless substrate (chromogen) to a colored product, indicating the presence of Ag:Ab binding. In a specific embodiment of the invention, a protein complex is based on B subunits of the heat labile enterotoxin (LT) of E. coli. In vivo, pentamerization of B subunits and binding of the pentamer to its natural receptor GM1 is the key event leading to uptake of the toxin and ultimately triggers the immonumodulatory events associated with mucosal immunity. As is exemplified herein, binding of a protein complex of the invention that is based on LTB subunits, is easily determined using GM1-ELISA as described by De Haan et al. (Vaccine 1996; 1:777-783). By using such type of an assay, it will be clear that an LTB-based protein complex of the invention, comprising at least one unaltered LTB subunit and at least one LTB-fusion protein, has retained its native pentamer formation.

In a further aspect of the invention, a host cell comprising a protein complex of the invention is provided. A host cell is for example a microbial cell provided with one or more nucleic acid construct encoding the components of a complex of the invention. In one embodiment, said host cell is a bacterial cell, for example a transformed E.coli cell. As mentioned above, a cell is preferably an edible cell comprising a protein complex capable of delivering one or more antigens to and across mucosal surfaces. Examples of edible cells are cells of edible plants, for example potato. Such a complex is advantageously used as a mucosal carrier molecule. Accordingly, an edible cell, or an extract thereof, comprising a mucosal carrier molecule of the invention can be used for oral vaccination e.g. via food or drinking water.

As said earlier, conventional uses of carrier molecules are essentially limited to the delivery of relatively small antigens. However, since a chimeric complex according to the invention has retained its ability to bind to a cell surface receptor, virtually irrespective of the size of the fused protein, it is now possible to use carrier molecules for the delivery of relatively large molecules of interest (antigens etc). It well known that, once an antigen is delivered to the appropriate site in the body, an immune response can be evoked. For example, antigens which are successfully delivered across the barrier of epithelial cells lining mucosal tracts stimulate underlying inductive sites of the mucosa-associated lymphoid tissue (MALT). The protein complexes of the invention can be provided with one or more antigenic molecules of interest, essentially without being limited to the size and complexity of the molecules of interest and, importantly, without loosing the carrier properties to deliver the complex across the epithelial barrier to stimulate the MALT. Therefore, a complex of the invention is advantageously used to induce an immune response in a subject, preferably a mammalian subject, such as a mouse or a human. In one embodiment, a chimeric protein complex of the invention comprising an antigen of interest is used in a vaccine. A vaccine comprising an complex of the invention with a particular antigen (or combinations of various antigens) is provided which, when administered to a subject, is capable of evoking an immune response that will protect the subject e.g. from an illness due to that antigen(s). The vaccine can be a therapeutic (treatment) vaccine which can be administered after infection and is intended to reduce or arrest disease progression. Preferably, it is a preventive (prophylactic) vaccine, capable of preventing initial infection of the subject.

Furthermore, a (plant-based) vaccine comprising a protein complex according to the invention or a cell comprising said complex is provided, as well as a pharmaceutical composition comprising an effective amount of said vaccine. A vaccine of the invention can be a multicomponent vaccine comprising a protein complex of the invention comprising at least two different antigens.

In yet another aspect of the invention, a method is provided for increasing an immune response of a subject to a specific pathogen which comprises administering, preferably orally, to the subject at least one dose of an effective amount of a protein complex of the invention, wherein the molecule of interest is an antigen. This method also opens the way to deliver protective antigens, including large (structural) antigens, to the immune system located in the intestinal tract upon oral delivery of the complex through feeding host (plant) cells or host cell compounds. Also provided herein is a method for mucosal (nasal, rectal or vaginal) immunisation comprising the administration of a vaccine of the invention to a subject via a preferred route of immunization. The invention thus provides an expansion of the size and variety of antigenic compounds that can be incorporated into carrier molecule-based vaccines

### LEGENDS

FIG. 1

Representation of the T-DNA part of the binary vectors pLANTIGEN4, 12, 13 and 15 containing the different LTB and LTB subunit vaccine gene constructs LB, left T-DNA border sequence; PNOS nopaline synthase promoter; NPTII, neomycin phosphotransferase II gene, selectable kanamycin resistance marker; TNOS, nopaline synthase terminator sequence; PPAT, class I patatin promoter; Gene, cloning site for expression under control of PPAT promoter; RB, right T-DNA border sequence; SP, signal peptide; LT-B, synthetic gene for LTB optimized for expression in plants; KDEL, endoplasmic reticulum retention signal; parvo, canine parvo virus (CPV) epitope; Ala, alanine; influenza, HA influenza virus hemagglutinin epitope; CSFV E2, classical swine fever virus EE2 glycoprotein lacking transmembrane domain.

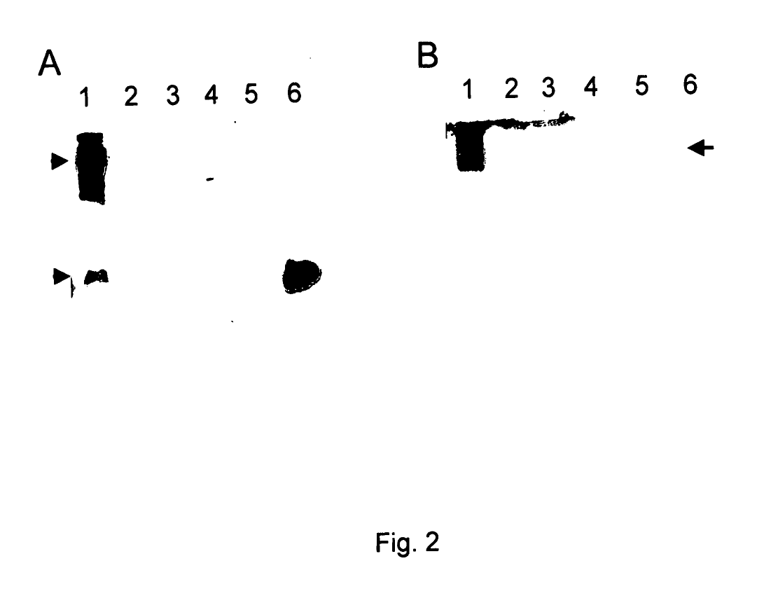

FIG. 2

Western analysis of tuber extracts (25 microgram total protein each). using LTB5 conformational monoclonal antibody VD12 (A) and CSFV E2 conformational mAb V3 (B). Lane 1, pL(4+13)16; lane 2, protein size marker; lane 3, control extract; lane 4, pL13(31); lane 5, pL13(17) and lane 6, pL4(17). Arrows left in A indicate LTB5 containing pentameric complexes and in B, CSFV E2 conformational epitope containing complex.

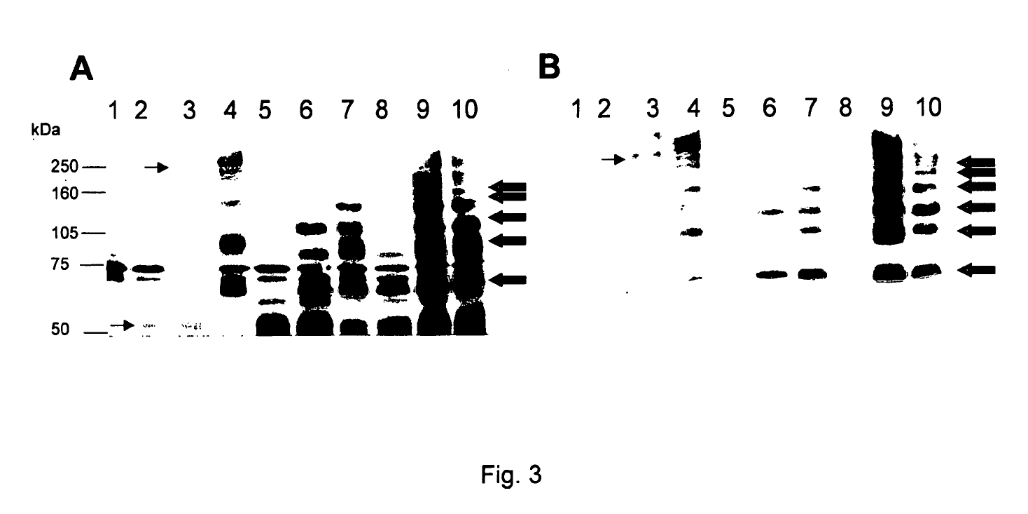

FIG. 3

conditions. A, left blot was incubated with VD12 (anti-LTB5) and B, right blot was incubated with V3 (anti-CSFV E;2). Fifty micrograms of total tuber protein each was loaded except for samples pL(4+13)16 (lane 4) and pL(4+13)46 (lane 7) of which 25 micrograms was loaded. Lane 1, wildtype tuber extract; lane 2, pL(4)21 extract; lane 3, pL(13)17 extract; lane 4, pL(4+13)16 extract; lane 5, pL(4+13)31 extract; lane 6, pL(4+13)39 extract; lane 7, pL(4+13)46 extract; lane 8, pL(4+13)60 extract; lane 9, pL(4+13)64 extract and lane 10, pL(4+13)67 extract. Arrows on the left of lane 2 indicate homopentameric (LTB)₅, lower arrow and homopentameric (LTB-CSFV E2)₅, upper arrow. Arrows on the right indicate chimeric complexes according to the invention. Molecular size marker is indicated at the left.

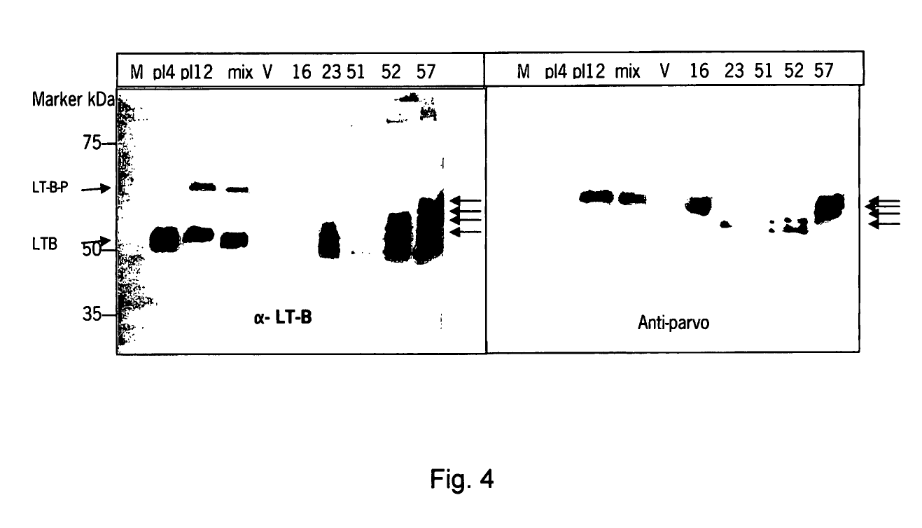

FIG. 4

Western blots of tuber extracts (25 microgram each) of pL(4+12) plants run on 12% SDS-PAGE gels under semi-native conditions. A, left blot was incubated with VD12 (anti-LTB5) and B, right blot was incubated with 3C9 (anti-parvo). Lane 1, M, full range rainbow molecular weight marker; lane 2, pL4 A(pL417) extract; lane 3, pL12 (pL(12)01) extract; lane 4, 1:1 mix of pL4 and pL 12 extracts (pL(4)17and pL(12)02); lane 5, V, PAT4 vector negative control; lanes 6-10, extracts of pL(4+12)16, pL(4+12)23, pL(4+12)51, pL(4+12)52 and pL(4+12)57, respectively. Arrows on the left depict LTB and LTB-parvo (LTB-P) and arrows on the right indicate chimeric complexes according to the invention. Solid dashes on the left indicate protein marker. The lower band-in lane 3 migrating slightly higher than LTB is a degradation product derived from LTB-P upon heating of the sample in loading buffer.

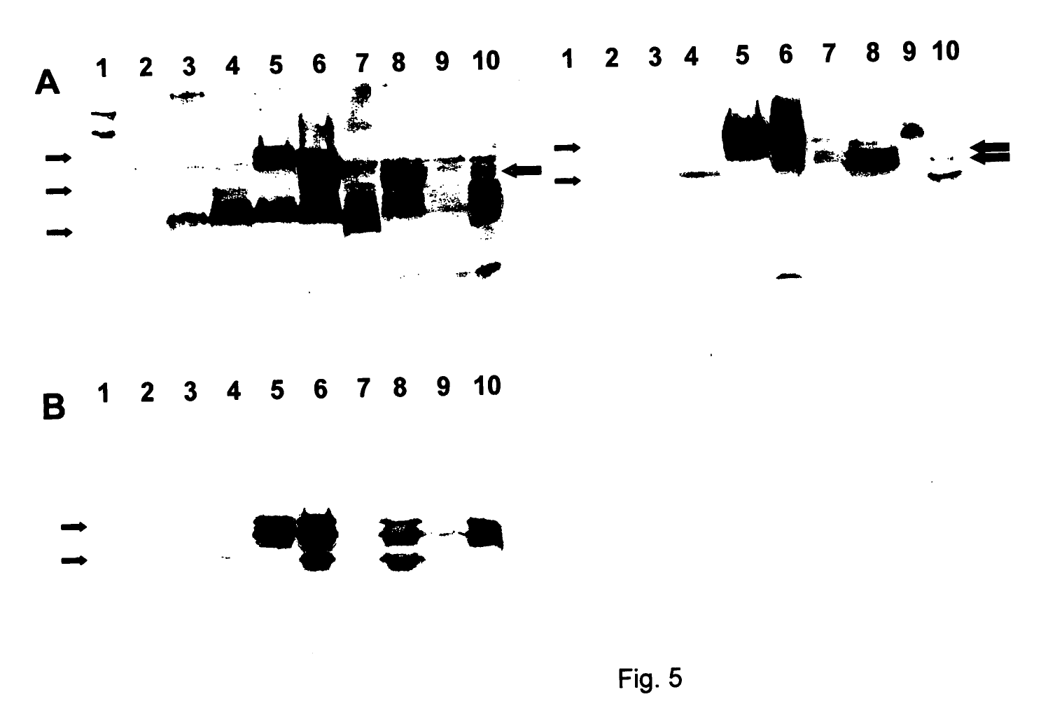

FIG. 5

Western blots of tuber extracts of pL(4+12+15) plants run under semi-native conditions on 10% SDS-PAGE gels (A) or reducing conditions on 15% SDS-PAGE gel (B).

A, left blot was incubated with VD 12 (anti-LTB5). and right blot with 3C9 (anti-parvo). B, blot was incubated with 3C9 (anti-parvo). 25 micrograms of total tuber protein extract each was loaded per lane. Lane 1, mol. Wt. standard; lane 2, wildtype tuber extract; lane 3, pL421 extract; lane 4, pL1201 extract; lane 5, pL1516 extract; lane 6-10, pL(4+12+15) tuber extracts: lane 6, pL(4+12+15)7; lane 7, pL(4+12+15)9; lane 8, pL(4+12+15)11; lane 9, pL(4+12+15)16 and lane 10, pL(4+12+15)19 extract. A, Arrows on the left indicate LTB5, lower arrow; (LTB-parvo)5 middle and (LTB-iipp)5, upper arrow. Arrows on the right indicate chimeric complexes B, lower arrow on the left indicates LTB-parvo and upper arrow, LTB-iipp.

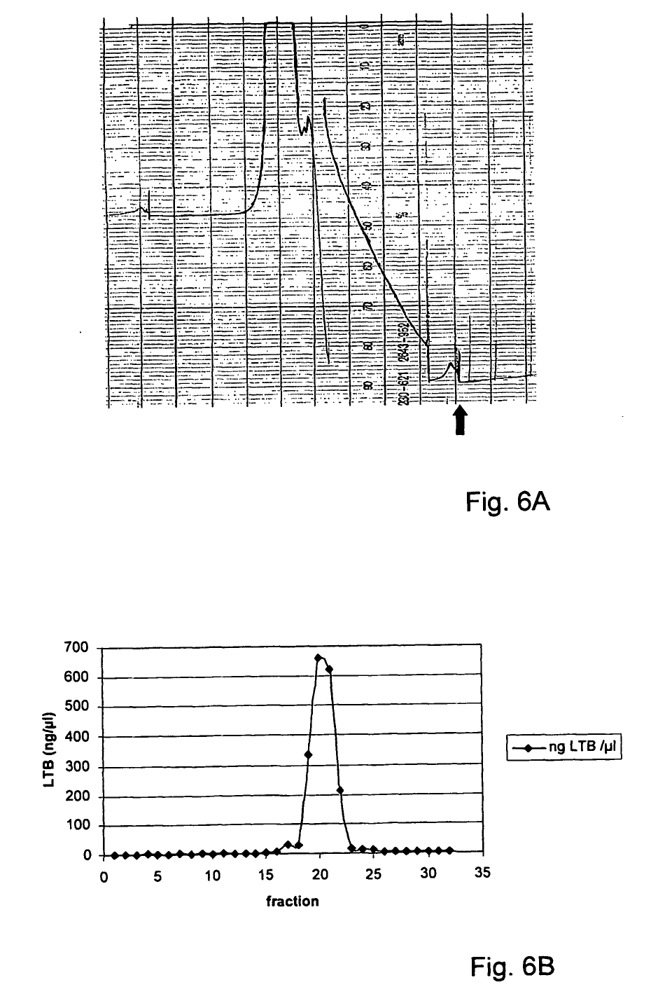

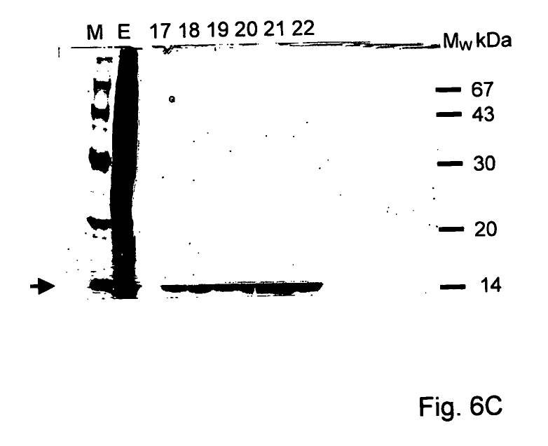

FIG. 6

Purification of Escherichia coli recLTB by affinity chromatography on immobilized D-galactose (Pierce). Supernatant from sonicated E.coli cells harvested by centrifugation, was loaded onto 5 cm D-galactose column fitted onto FPLC apparatus after extensively washing of the column with 6 vols. TEAN buffer. Crude protein extract from E.coli dissolved in 47.5 mL TEAN buffer to which protease inhibitor cocktail was added (Roche), was loaded onto column at flow 0.5 mL/min. Washing was with three volumes (142.5 mL) TEAN buffer (50 mM Tris-HCl, pH7.4; 0.2 M NaCl; 1 mM EDTA) at 0.5 mL/min. Elution was with washing buffer supplemented with 0.5 M D-galactose at 0.5 mL/min. Fractions of 1 mL were collected and placed on ice immediately after collection. Detector was set at 0.1 A.U. and recorder speed 0.25 cm/mL.

A. Elution profile. Solid arrow depicts start elution TEAN buffer supplemented with 0.5 M D-galactose. Solid line under record represents fractions nr. 16-22 (major peak).

B. GM1-ELISA pooled fractions 1-32 (1 mL each). The amount of LTB5 (ng/μl fraction) was established by GM1 ELISA.

C. Coommassie stained gel fractions 17-22. A pre-cast 10% SDS-PAGE gel (Bio-Rad) was run under reducing conditions. Lane M, molecular size marker; lane E, 10 μl crude extract (before column); lanes 17-22, 10 μl each fraction. Samples were boiled for 3 min prior to loading and running was at standard conditions. Gel was stained with Coommassie Brilliant Blue overnight and destained with 0.3% Tween-20. Arrow at the left depicts LTB monomer and sizes molecular weight marker are indicated at the right (kDa).

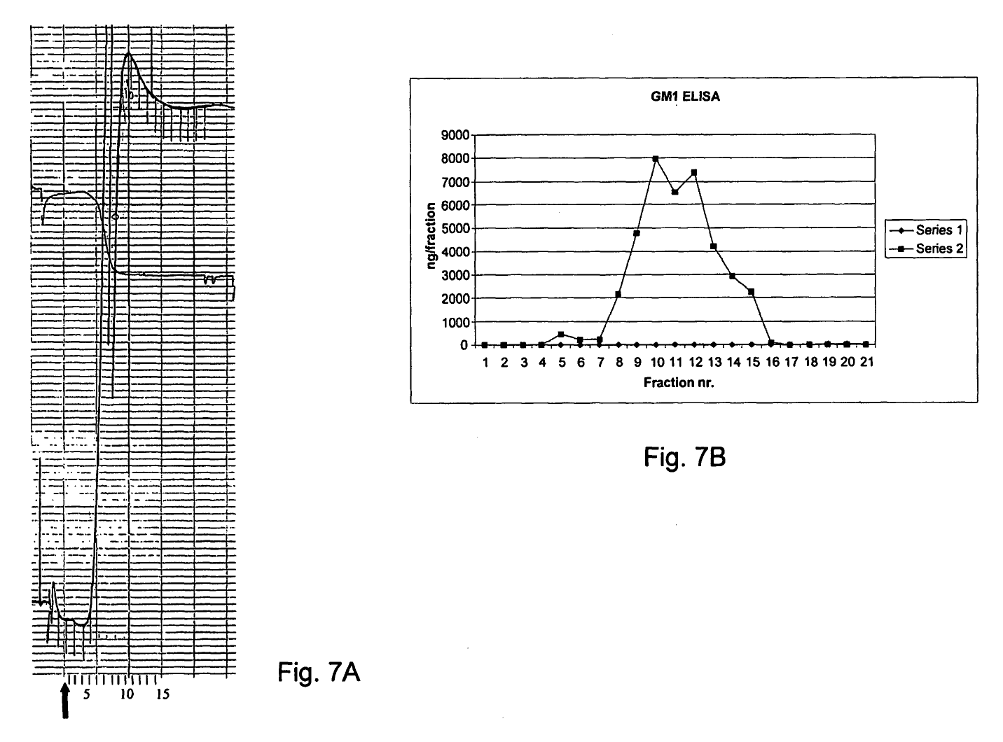

FIG. 7

Purification of recLTB from 38 grams fresh weight tuber of pL421 plant by affinity chromatography on immobilized D-galactose (Pierce). Tuber was peeled and cut into small pieces and 70 mL cold extraction buffer was added and grinding and extraction was performed in a stainless steel blender. The supernatant containing the crude protein extract and recLTB was centrifuged for 5 min at 15300 rpm at 4° C. to remove particles and starch granules. The centrifugation step was repeated until the supernatant was completely clear and the remaining 47 mL crude extract was loaded onto the column. Loading was at 0.5 mL/min and the column was cooled at 4° C. After loading the column was washed with 42 mL TEAN buffer at 0.75 mL/min and elution was with TEAN buffer supplemented with 0.3 M D-galactose (instead of 0.5 M; 26 mL total). Fractions were collected. Fraction size was 0.75 mL and fractions were placed on ice immediately after collection Detector was set at 0.2 A.U. and recorder speed 0.25 cm/mL.

A. Elution profile. Solid arrow depicts start elution TEAN buffer supplemented with 0.3 M D-galactose. Solid dashes under record represent 0.75 mL fractions nr. 1-15. Major peak was at fraction 10-11.

B. GM1-ELISA fractions 1-21 (0 75 mL each). The amount of LTB5 (ng/fraction) was established by GM1ELISA.

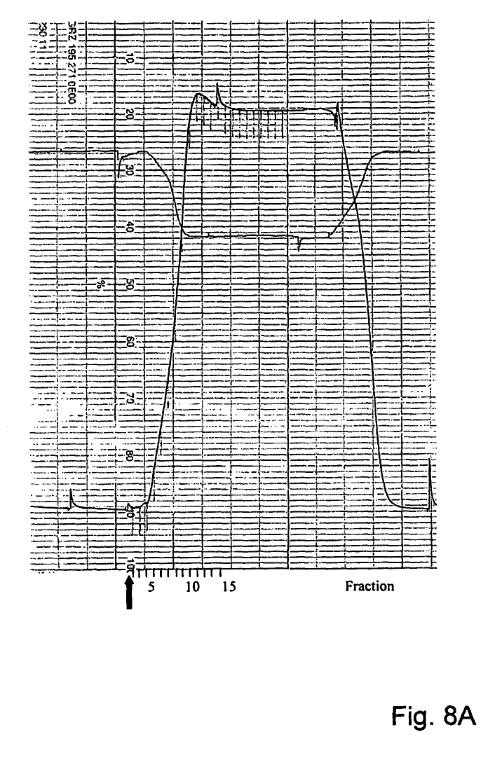

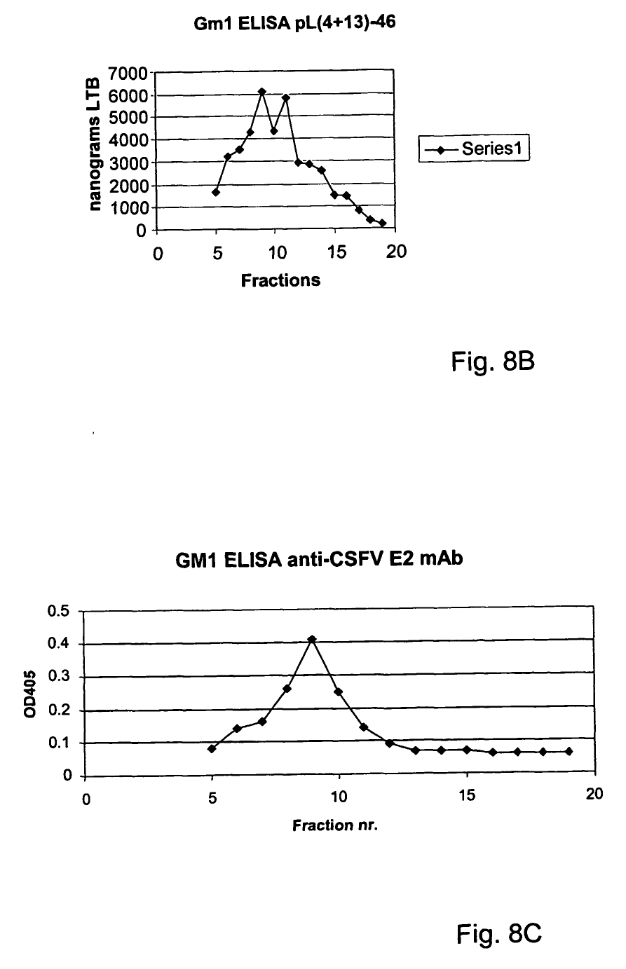

FIG. 8

Purification of the chimeric protein complex pL(4+13) from 6.5 grams of freeze-dried tuber of pL(4+13)46 plant by affinity chromatography on immobilized D-galactose (Pierce). Prior to extraction, 26 mL water was added to the freeze-dried tuber material followed by 65 mL extraction buffer supplemented with protease inhibitor cocktail (Roche). Extraction was under continuous shaking for 22 min on ice and extract was filtered through a 80 μM mesh cloth and centrifuged twice at 4° C. for 10 min at 15300 rpm to remove remaining starch granules. The remaining 57 mL clear crude extract was loaded onto the column. Loading was at 0.5 mL/min and the column was cooled at 4° C. After loading the column was washed with TEAN buffer at 0.75 mL/min for 61 min and elution was with TEAN buffer supplemented with 0.3 M D-galactose. Fractions were collected. Fraction size was 0.75 mL and fractions were placed on ice immediately after collection. Detector was set at 0.2 A.U. and recorder speed 0.25 cm/mL.

A. Elution profile. Solid arrow depicts start elution TEAN buffer supplemented with 0.3 M D-galactose. Solid dashes under record represent 0.75 mL fractions nr 1-15. Major peak was at fraction 10-11.

B. Standard GM1-ELISA fractions 5-19 (0.75 mL each). The amount of LTB5 (ng/fraction) was established by GM1 ELISA.

C. Modified GM1-ELISA fractions 5-19. Detection of binding was with V3 mAb specific for CSFV E2 and alkaline-phosphatase labeled sheep-anti-mouse IgG (instead of VD12, anti-LTB5).

### EXAMPLES

### Example 1

### Construction of LTB Subunit Vaccine Expression Cassettes

A schematic overview of the T-DNA part of the binary plant expression vector pBINPLUS (Van Engelen et al., 1995) and the gene inserts of all the pLANTIGEN vaccine constructs reported here, is represented in FIG. 1. All genes were placed under control of the class I patatin promoter (Ppat) for expression in tubers only and in addition harbour a DNA sequence that codes for a KDEL (Lys-Asn-Gln-Leu) sequence at the C-terminus of the respective fusion proteins for retention in the ER (Munro and Pelham, 1987).

pL4: The design and construction of at synthetic gene for LTB (synLT-B) and the generation of the binary plant expression vector pLANTIGEN4 (pL4) was described before (Lauterslager et al., 2001). pL4 harbours the synthetic gene for LTB (synLT-B) with a unique BamHI restriction site just after the sequence coding for the mature LTB protein and preceding the sequence coding for KDEL All synthetic sequences were made in such a way and cloned in this unique site, that all were in frame with LTB and the KDEL sequence at the carboxy terminus.

pL12: The core of the fragment coding for the canine parvo virus (CPV) epitope cloned in pLANTIGEN12 (pL2); codes for the amino acid sequence SDGAVQPDGGQPAVRNERAT (Langeveld et al., 1994). pLANTIGEN12 was made by cloning a synthetic BamHI/BglII fragment coding for the amino-terminal region of the viral protein VP2 of canine parvovirus (CPV) into the unique BamHI site of pL4. The synthetic fragment was made by ligation of fragments derived from oligo's as described before (Florack et al., 1994). Oligo's were from Eurogentec (Belgium).

pL13: In pLANTIGEN13 (pL13) a fragment coding for the CSFV E2 glycoprotein lacking the C-terminal transmembrane (TM) region was ligated. In wildtype CSFV the E2 glycoprotein is transmembrane bound. pL13 was constructed by cloning a BamHI fragment coding for the E2 mature protein of the classical swine fever virus (CSFV) into the unique BamHI site of pL4. The fragment coding for CSFV E2 was obtained by PCR of pPRb2 (Hulst et al., 1993) using oligos 5′-gttcatccttttcactgaattctgcg-3′ and 5′-cgcagaattcagtgaaaaggatgaac-3′.).

pL15: In pLANTIGEN15 (pL15), the CPV sequence was cloned twice together with a doubled HA epitope sequence, each separated by two alanine residues for spacing. The HA epitope codes for the decapeptide FERFEIFPKE and represents amino acids 111-120 of PR8 HA-1 (Hackett et al., 1985). CPV is a linear B cell epitope whereas HA is T cell specific. pL15 was constructed by cloning a synthetic fragment coding for a tetrameric sequence consisting of a doubled decapeptide of influenza virus hemagglutinin (HA) heavy chain together with a doubled CPV epitope similar to what was cloned in pL12, into the unique BamHI site of pL4. All four epitope sequences were cloned in such a way that they were separated by two alanine residues each.

### Example 2

### Host Cell Transformation, Growth and Protein Extraction

Previously we have described the generation of 22 independent transgenic potato plants containing the pL4 gene constructs which resulted in 16 lots of tubers (Lauterslager et al., 2001). Since then, more transformation experiments were conducted which yielded additional 31 independent tuber lots harbouring the pL4 gene construct.

In the present invention, binary expression vectors described in Example 1, and combinations thereof, were introduced in Agrobacterium tumefaciens strain AgI0 (Lazo et al., 1991) by electroporation and used for transformation of Solanum tuberosum cultivar Désirée (De Z. P. C., Leeuwarden, The Netherlands). Transformation, growth, selection of transgenic shoots and tuber production are described previously (Lauterslager et al., 2001). Transformation of stem internodes of potato cultivar Désirée with pL12 generated 27 independent transgenic plants of which 22 produced tubers in the greenhouse. Transformation with pL13 generated 23 plants, of which 20 formed tubers. Transformation with pL15 yielded 31 plants, of which 20 produced tubers.

Typically, 200 to 300 grams of tubers were harvested after 2-4 months from greenhouse grown plants. Extracts were made from freshly harvested tuber material of approximately the same size to reduce effects caused by storage or tuber age. For the isolation of a protein complex produced by the potato host cell, freshly harvested tubers of approximately 5 cm diameter were used. Skinless tuber slices were extracted in 25 mM sodium phosphate pH 6.6, 100 mM NaCl, 1 mM ethylenediamine tetraacetic acid (EDTA), 50 mM sodium ascorbate, 1% Triton X-100 and 20 mM sodium metabisulphite. Tissue homogenate was centrifuged at 4° C., 12000 rpm for 5 min and supernatant was collected and transferred to a fresh tube. Total soluble protein was estimated by the method of Bradford (Bradford, 1976) using bovine serum albumin (BSA) as standard. Expression or accumulation of LTB pentameric complexes was confirmed by GM1-ELISA (Example 3).

### Example 3

### Determining Receptor Binding of a Protein Complex

The amount of functional chimeric protein complexes isolated from transgenic potato tubers was estimated by a modified ganglioside GM1enzyme-linked immunosorbent assay (GM1-ELISA). Microtiter plates (PolySorp Immunoplates, Nunc) were coated with monosialoganglioside GM1 from bovine brain (Sigma Aldrich, St. Louis, USA) at 5 μg/mL in phosphate buffered saline (PBS). Fixed amounts, typically 5 μg of total soluble, extractable tuber protein, were loaded onto coated plates, pretreated by washing three times with deionized water and blocked for 1 h at room temperature with 2% skimmed milk, 0.1% bovine serum albumin and 0.1% Tween-20 in PBS under continuous shaking at 100 rpm. A serial twofold dilution of recombinant LTB produced by an Escherichia coli expression vector was added to each plate spiked with equal amounts of total protein from pBINPLUSPAT tubers. Binding was allowed for 16 h at 4° C. After washing plates three times with deionized water, plates were incubated with monoclonal antibody VD12, specific for LTB pentamers, for 1 h at room temperature in a 1:1000 dilution. After rinsing with deionized water three times, plates were further incubated with AP-labelled sheep-anti-mouse antibody and bound label was detected with 4-nitrophenylphosphate (disodium salt hexahydrate; Janssen Chimica). Detection was at 405 nm in a Bio-Rad Benchmark Microplate Reader (Bio Rad, Veenendaal, The Netherlands) using Microplate Manager/PC version 4.0 software for standard curve analysis, calculation of concentrations and standard deviation. The amount of pentameric LTB was estimated by comparison of the readings of samples comprising isolated pentamers with the results from the standard curve. Samples were analysed at least twice in independent experiments. For the calculation of molar rates, the amount of total protein per gram fresh weight of tuber was set at 7 mg/g. The molar weight of the fusion proteins was deduced from the known amino acid sequences of the mature proteins encoded by the respective pLANTIGEN fusion constructs. The average expression level of the pentameric protein complexes produced in transgenic tubers are presented in Table 1. These data show that, the larger the size of the molecule of interest, the lower the expression level of functional (i.e. GM1-binding) homopentameric protein complex.

### Example 4

### Chimeric Protein Complex Comprising LTB and LTB-CSFV E2

Binary expression vectors pL4 (Lauterslager et al., 2001) encoding an unaltered subunit and pL13 (LTB-CSFV E2 fusion; for overview see Example 1) encoding a molecule of interest fused to a subunit were introduced in potato by co-transformation using Agrobacterium tumefaciens mediated transformation of Solanum tuberosum cv. Désirée (De Z. P. C., Leeuwarden, The Netherlands) essentially as described before (Lauterslager et al., 2001) and outlined above (Example 1). To enable co-transformation, prior to infection of stem internodes both recombinant Agrobacteria harbouring pL4 and pL13 were mixed in a 1:1 ratio at OD595=1. The mixed bacterial suspension was used in transformation experiment and regeneration of transformed cells and selection of transgenic shoots was as described before. Seventy-one independent transgenic shoots were obtained and 53 selected and analysed for the presence of pL4 and/or pL13 gene constructs to reveal co-transformed events which were selected for further analysis. To this end, genomic DNA was isolated from leaf material collected of individual plants by grinding leaf discs of approximately 5 mm diameter in 50 μl urea extraction buffer (62% ureum, 0.5 M NaCl, 70 mM Tris-HCl pH8.0, 30 mM EDTA pH 8.0, 1.5% sarkosyl). An equal volume of phenol/chloroform (1:1) was added and samples were mixed and left at room temperature for 15 min. After mixing, samples were centrifuged for 10 min at 3000 rpm, supernatants were transferred to fresh tubes and 10 μl 4.4 M ammonium-acetate pH 5.2 was added. To precipitate the genomic DNA 120 μl isopropanol was added and mixed. Samples were centrifuged for 3 min at 1000 rpm and supernatant removed. The remaining pellets were dried and suspended in water or buffer containing 10 mM. Tris-HCl pH8.0 and 1 mM EDTA. Microliter amounts of the genomic DNA samples were submitted to PCR using specific primers that can distinguish pL4 and pL13 gene construct such as but not limited to primers LTB11 (5′-ggtgatcatcacattcaagagcggtgaaacatttcaagtc-3′) and Tnosminus50 (5′-atgataatcatcgcaagaccg-3′). Amplification conditions were: 40 cycles, each 94° C. for 30 seconds to enable denaturation, annealing at 56° C. for 45 seconds followed by elongation at 68° C. for 2 minutes, using AccuTaq polymerase (Sigma-Aldrich) at optimal conditions according to the manufacturer PCR reaction mixtures were submitted to 1.2% agarose gel electrophoresis in 0.5× TBE and gels were scanned for the presence of fragments corresponding to the amplified gene constructs of pL4 and/or pL13 for which the predicted sizes are well known and were deduced from their known gene sequences and the use of primers LTB11 and Tnosminus50. Twenty-two plants contained both gene constructs, whereas twenty-eight only contained pL4 and, remarkably, only three had pL13 gene construct. The 22 plants that were positive for both the pL4 and pL13 gene constructs, such as plant number 8, herein further referred to as pL(4+13)8, or plant pL(4+13)16, pL(4+13)31, pL(4+13)39 or pL(4+13)46 were transferred to the greenhouse and grown to maturity for the production of tuber material for further analysis of accumulation of chimeric protein complex.

Tubers were harvested and the amount of GM 1-binding LTB pentamers was evaluated as described in Example 2 and 3. Several of the plants showed a significant accumulation of GM1-binding LTB pentamers, for example plants pL(4+13)15, pL(4+13)16, pL(4+13)39, pL(4+13)46 pL(4+13)60, pL(4+13)64 and pL(4+13)67 (see Table 2). These plants were characterized further Tuber material of selected co-transformed potato plants accumulated LTB5 pentamers as deduced from binding to ganglioside GM1, such as pL(4+13)16 and pL(4+13)46 at approximately 8 and 20 micrograms per gram fresh weight FW) tuber, respectively (Table 2).

Chimeric plant pL(4+13)16 was further evaluated by Western blotting. To this end, twenty-five micrograms of total protein of tuber extracts a plant was loaded onto a 10% SDS-PAGE gel and run under semi-native, non-reducing conditions to enable separation of protein complexes. Following semi-native SDS-PAGE, separated proteins were blotted onto nitrocellulose using standard techniques in CAPS (0.22% 3-[cyclohexylamino]-1-propane-sulfonic acid pH11 in 10% ethanol) buffer for subsequent Western analyses using specific antibodies; either VD12 which is an anti-LTB5 conformational monoclonal antibody (FIG. 2A), or V3 which is an anti-CSFV E2 conformational monoclonal antibody, at 1:1000 dilution (FIG. 2B). Specific antibody binding/recognition was visualized using a horseradish peroxidase labelled secondary antibody and LumiLight substrate and visualized in a LumiImager (Roche, Boehringer, Germany). As controls pL1317 and 1331 were used, both harbouring pL13 gene construct pL1317 was previously identified as the highest expressor of GM1-binding pentamers for LTB-CSFV-E2 fusion protein amongst twenty independent pL13 transgenic plants. Furthermore, pL417 harbouring the pL4 gene construct and accumulating approx. 17 microgram/g FW GM1-binding LTB pentamers and a negative control for GM1-binding pentamers were used.

FIG. 2A visualizes the results of the analysis of 25 microgram amounts of total protein extracts of these plants using the conformational anti-LTB5 monoclonal antibody VD12 and FIG. 2B upon analysis with V3, specific for conformational CSFV E2. Loaded were co-transformed plant pL(4+13)16 (lane 1; co-transformed with pL4 and pL13), protein molecular weight marker (lane 2), a control extract harbouring PAT4, an empty expression cassette vector (lane 3), extracts of pL1331 (lane 4) and pL1317 (lane 5), both harbouring the LTB-CSFV E2 gene fusion only, and pL(4)17 (lane 6) transformed with pL4 and only accumulating the unaltered rec-LTB subunit.

From FIG. 2 it is apparent that pL(4+13)16 accumulates a significant amount of protein complexes that are significantly larger in size than homo pentameric LTB5 (compare lanes 1 and 6) and that are recognized by both conformation specific monoclonal antibodies VD12 and V3 (compare lane 1 in A and B). This demonstrates the existence of a chimeric protein complex harbouring a pentameric LTB5 structure recognized by VD12 as well as an antigenic CSFV E2 dimeric epitope recognized by V3. Importantly, the latter antigenic CSFV E2 dimeric epitope is nearly absent in pL13 (lanes 4 and 5), which only comprises the LTB-CSFV E2 fusion protein and no unaltered LTB subunit. In addition, the approx 300 kDa homopentameric LTB-CSFV E2 complex is hardly visible after analysis with VD12 and V3. Conceivably, this is due to its very low expression level as observed before. From FIG. 2 and GM1 ELISA it is also apparent that only a chimeric complex comprising both LTB (pL4) and LTB-CSFV E2 (pL13), as present in plant pL(4+13)16 (FIGS. 2A and B, lane 1), facilitates the accumulation of a significant amount (8 micrograms per gram FW tuber) of a protein complex that harbours both GM1-binding properties and an antigenic CSFV E2 epitope. In contrast, a plant expressing only the LTB-CSFV E2 gene fusion as for instance plant pL1317 does not form an antigenic protein complex capable of binding the GM1 cell surface receptor (compare lanes 1 and 4 in FIGS. 2A and B).

Chimeric plants pL(4+13)16, pL(4+13)31, pL(4+13)39, pL(4+13)46, pL(4+13)60, pL(4+13)64 and pL(4+13)67, all accumulating significant amounts of functional LTB5 according to GM1ELISA (Table 2), were further evaluated by Western blotting as described. FIG. 3A shows the results after incubation with VD12 and indicates the presence of multiple chimeric complexes comprising LTB5 in extracts of pL(4+13)16 to pL(4+13)67, as is apparent from its migration to a position that is between that of pL4(21) (lane 2) containing only LTB5 and pL13(17) (lane 3) containing (LTB-CSFV E2)5. As expected, these chimeric complexes also react with V3 mAb which is specific for CSFV E2 (panel B, FIG. 3). From FIG. 3B it also clear that extracts from chimeric plants (lanes 6-10). react stronger with V3 mAb although similar amounts of total protein were loaded compared to pL13(17) (lane 2), further underscoring the accumulation of functional CSFV E2 dimers on GM 1-binding LTB5 complexes.

### Example 5

### Chimeric Protein Complex Comprising LTB and LTB-Parvo

Binary expression vectors pL4 (LTB) and pL12 (LTB-CPV parvo; for overview see FIG. 1 and Example 1) were both introduced in potato by co-transformation using Agrobacterium tumefaciens mediated transformation of Solanum tuberosum cv. Désirée essentially as described in Example 4. Prior to infection of stem internodes, recombinant Agrobacteria harbouring either pL4 or pL12 were mixed in a 1:1 ratio at OD595=1 and the mixed bacterial suspension was used in transformation experiment to enable co-transformation. More than forty-five independent transgenic shoots were selected and analysed for the presence of pL4 and/or pL12 gene constructs to reveal co-transformed events which were selected for further analysis. Twenty transgenic plants appeared to contain both gene constructs whereas nineteen plants had only pL4, and six plants had only pL12. All twenty double transformants were grown in the greenhouse for tuber formation and tubers were harvested and the amount of GM1-binding LTB pentamers established. A summary of selected pL(4+12) plants is given in Table 3.

Western blotting (FIG. 4) using VD12 and 3C9 a mAb specific for canine parvo virus indicated that several plants, such as pL(4+12)19, pL(4+12)20, pL(4+12)23, pL(4+12)31, pL(4+12)41, pL(4+12)46, pL(4+12)51, pL(4+12)52, pL(4+12)57, pL(4+12)62 and pL(4+12)65 contained chimeric protein complexes migrating on the SDS-PAGE gels in between LTB5 (pL4, lane 2) and LT-CPV5 (pL12, lane 3). From FIGS. 4A and B it is apparent that the protein extracts of some plants, such as pL(4+12)31, pL(4+12)57 and pL(4+12)62, exhibited multiple bands that were recognized by both VD12 (anti-LTB5) and 3C9 (anti parvo) indicating the presence of chimeric complexes, e.g. containing either 4 unaltered LTB subunits and 1 LTB-CPV fused subunit, or 3 LTB and 2 LTB-CPV, or 2 LTB and 3 LTB-CPV or 1 LTB and 4 LTB-CPV (FIGS. 4A and B). Especially in the extract of pL(4+12)57 it is clear that there are four chimeric complexes besides homopentameric LTB5 and homopentameric (LTB-parvo)5.

### Example 6

### Chimeric Protein Complex Comprising LTB, LTB-Parvo and LTB-iipp

Binary expression vectors pL4 (LTB), pL12 (LTB-CPV parvo) and pL15 (LTB-iipp iipp=influenza-influenza-parvo-parvo, double influenza epitope combined with double parvo epitope; for overview see Example 1) were introduced in potato by co-transformation using Agrobacterium tumefaciens mediated transformation of Solanum tuberosum cv. Désirée essentially as described above. Twenty-nine independent transgenic shoots were selected and analysed for the presence of pL4, pL12 and pL15 gene constructs to reveal co-transformed events. These plants were selected for further analysis. Five transgenic plants appeared to contain all three gene constructs, whereas ten contained two gene constructs, either pL(4+12), pL(4+15) or pL(12+15). The remaining 14 plants contained only one gene construct. All plants harbouring more than one gene construct were transferred to the greenhouse and grown to maturity. All five triple transformants, pL(4+12+15)7, pL(4+12+15)9, pL(4+12+15)11, pL(4+12+15)16 and pL(4+12+15)19, were analysed by GM1 ELISA and Western blotting using VD12 and 3C9 mAbs (FIG. 5).

From FIG. 5A left panel it is apparent that the five plants comprising all three gene constructs (lanes 6 to 10) contain complexes that migrate in between those of pL(4)21 (lane 3) and pL(12)01 (lane 4) or between pL(12)01 (lane 4) and pL(15)16 (lane 5) suggesting various combinations of LTB, LTB-parvo and/or LTB-iipp. From FIG. 5A right panel it is apparent that these complexes react positively with mAb 3C9, indicating the presence of at least one parvo epitope. From FIG. 5B it is clear that especially pL(4+12+15)7 (lane 6) and pL(4+12+15)11 (lane 8) contain both LTB-parvo and LT B-iipp. Hence, in these plants a chimeric GM1 binding protein complex is produced that contains LTB, LTB-parvo and LTB-iipp in one.

### Example 7

### Construction of E.coli LTB Subunit Expression Cassettes

A protein complex according to the invention can be produced in various kinds of recombinant host cells. Examples 7 and 8 describe the production of a chimeric protein complex comprising at least one unaltered subunit and at least one LTB subunit fused to a molecule of interest, in this case the reporter molecule GFP, in the micro organism E. coli.

For expression in Escherichia coli and other prokaryotes the original wildtype E.coli sequences for LTB (EtxB) were used. The LTB coding sequence was cloned from pYA3047 [Jagusztyn-Krynicka et al., 1993] which greatly resembles the nucleotide sequence ECELTBP (SWISS-PROT P32890) originally isolated from a porcine E.coli strain (Dallas and Falkow, 1980; Leong et al., 1985). A fragment was amplified by PCR using primers LTBbpi (5′-GTGACGAAGACAACATGAATAAAGTAAAATGTTATGTT-3′) and LTBbameco (5′-GTGACGAATTCTATGGATCCCCTGGAGCGTAGTTTTCATACTGATTGCC-3′) and a vector comprising wildtype EtxB sequence as template. The resulting BpiI/EcoRI was cloned in a pET21d vector Novagen) digested with NcoI/EcoRI generating pET-wiLTB1. After verification of nucleotide sequence, the resulting clone was transformed into TOP10F° cells (Invitrogen) for expression studies. An LTB-GFP fusion protein was made by introducing BamHI/BpiI sites at the termini of GFP sequence by PCR using primers GFPbam (5′-GTGACGGATCCGGCTTCCAAGG-3′) and LTBGFPbam (5′-GTGACGAAGACAAGATCTTACTTGTACAACTCATCCA-3′) and cloned into pET-wiLTE digested with BamHI After verification of nucleotide sequence, resulting clone pET-wiLTB-GFP2 was transformed into TOP10F° cells for expression studies (Example 8).

### Example 8

### E.coli Cell Transformation, Growth and Protein Extraction

Two selected clones, pET-wiLTB1 and pET-wiLTB-GFP2 were transformed into E.coli Rosetta strain (Novagen) and grown overnight at 37° C. in LB medium supplemented with 50 mg/L ampicillin and 34 mg/L chloramphenicol 2 mL of o/n culture was diluted into 50 mL fresh medium and grown at 20° until OD595 reached 0.2 with continuous shaking. Expression induced by adding IPTG to final concentration 1 mM and culture was further grown to OD595=0.5. Cells were collected by centrifugation for 10 min at 14000 rpm at 4° C. and pellet was resuspended in BugBuster (Novagen) Extraction Reagent and lysozyme was added up to final concentration 1 μg/μl. Samples were incubated at room temperature for 5 min and Benzonase was added and further incubated for 15 min at room temperature. Samples were subsequently centrifuged for 5 min at 4° C. at 14000 rpm. The supernatant of pET-wiLTB was loaded onto an immobilized D-galactose column for purification of LTB5 (see also Example 10). The elution profile is depicted in FIG. 6. From FIG. 6A it is apparent that immediately upon applying elution buffer protein can be measured in the respective fractions. From FIG. 6B it is clear that these fractions contain GM1-binding LTB5 complexes as apparent from GM1 ELISA RecLTB-GFP appeared to accumulate in inclusion bodies. The resulting pellet containing inclusion bodies was further treated essentially as described by Sambrook et al. (1989) with minor modifications according to Khoury and Meinersmann (A genetic hybrid of the Campylobacter jejuni flaA gene with Escherichia coli and assessment of the efficacy of the hybrid as an oral vaccine. Avian disease 39 (1995) 812-820). Inclusion bodies were isolated using standard technologies and extracted with 8 M Urea. The urea soluble fraction was dialyzed against 0.1 M Tris (pH 7.4), and the precipitating fraction was removed by centrifugation for 10 min at 10000 g. The supernatant containing GM1-binding complexes was further purified by affinity chromatography on D-galactose to obtain purified recLTB-GFP.

### Example 9

### Production of LTB and LTB-GFP Chimeric Complex

An alternative method to produce a chimeric complex according to the invention comprising at least one unaltered LTB subunit and at least one LTB-GFP fused subunit involves providing a first composition comprising the unaltered subunit and a second composition comprising the fused subunit, followed by mixing both compositions in a 1:4, 2:3, 3:2 or 4:1 molar ratio. Subsequently, the mixture of both types (i.e. unaltered and altered) of subunits is de- and renatured under pentamer inducing conditions. For example, recLTB and recLTB-GFP produced in two strains of recombinant E.coli as described in Examples 7 and 8 and purified by affinity chromatography on D-galactose were dissolved in 8 M Urea. Alternatively, purified recLTB can be added to inclusion bodies comprising recLTB-GFP and further treated as described above. The urea-soluble fraction can be dialysed against 0.1 M Tris (pH 7.4) and insoluble material removed by centrifugation at 10000 g for 10 min at 4° C. Alternatively, 0.3 M D-galactose can be added to the Tris buffer to promote pentamerization. The supernatant containing pentameric complexes comprising chimeric LTB and LTB-GFP molecules can be further purified by affinity chromatography, e.g. on immobilized D-galactose. The chimeric subunit composition of the protein complexes can be verified by semi-native SDS-PAGE and Western blotting as described above GM1-binding can be determined using GM1-ELISA as described above.

### Example 10

### Purification of LTB Subunit Carrier Complexes

Homopentameric RecLTB5 and LTB-subunit carrier complexes were purified by affinity chromatography on immobilized D-galactose agarose (Pierce, cat. no. 20372) according to Uesaka et al. (Uesaka et al. (1994) Simple method of purification of Escherichia coli heat-labile enterotoxin and cholera toxin using immobilized galactose. Microbial Pathogenesis 16: 71-76).

E.coli cells provided with a microbial LTB-expression cassette as described in Example 7 were grown as described in Example 8 and collected by centrifugation at 6500 g for 30 min at 4° C., sonicated and further treated as described (Uesaka et al., 1994). Supernatants from sonicated E.coli were adjusted to 50 mM Tris-HCl pH 7.4, 0.2 M NaCl, 1 mM EDTA and 20 mM sodiummetabisulphite (TEAN) and loaded onto the D-galactose column fixed to an FPLC (BioRad, Veenendaal, The Netherlands). The column was washed with TEAN buffer and elution was with TEAN buffer containing either 0.3 M D-galactose or 0.5 M D-galactose according to standard procedures known to persons skilled in the art. FIG. 6 shows the result of the purification recLTB from E.coli. Fractions were collected and the presence of GM1-binding LTB was determined by GM1-ELISA as described in Example 3. The results for the purified recLTB from E.coli as depicted in FIG. 6A is given in FIG. 6B. All fractions positive for protein contained GM1-binding complexes as apparent from GM1-ELISA The elution profile for the purification of recLTB from E.coli on immobilized D-galactose and analysis of the presence of GM1 binding activity by GM-1 ELISA of corresponding fractions is presented in FIG. 6.

Next, protein complexes were purified from the supernatant of tuber extract from pL4(21) by D-galactose chromatography (FIG. 7). From FIGS. 7A and B it is clear that such complexes can also be purified from tubers.

Next, chimeric protein complexes were purified from a pL(4+13) plant. Extracts were prepared from pL(4+13)46 tuber material by grinding tuber material in extraction buffer as described in Example 2 and extracts were centrifuged at 6500 g for 30 min at 4′C. to remove insoluble material and starch granules (FIG. 8). The supernatant was adjusted to TEAN buffer conditions and loaded onto the D-galactose column and further treated as described. From FIG. 8 it is clear that proteins are eluting from the column starting with fraction 5 with a maximum absorbance in fractions 10-11. From GM1-ELISA it is also apparent that the majority of LTB as detected with VD12 mAb is in fractions 5-15 with a max in fraction 10 (FIG. 8B). In addition, these fractions are also positive for CSFV E2 epitope as apparent from a modified GM1 ELISA in which the second antibody was the conformational anti-CSFV E2 mAb V3 (FIG. 8C) indicating the presence of both LTB and LTB-CSFV E2 in such complexes which is in agreement with previous results obtained from Western blotting (FIG. 3).

### Example 11

### Chimeric Complex of LTB and LTB-VHSV G

A genetic fusion of LTB and the spike glycoprotein G from viral hemorrhagic septicemia virus similar to sequence X66134 (EMBL) and published by Lorenzen et al. (Molecular cloning and expression in Escherichia coli of the glycoprotein gene of VHS virus, and immunization of rainbow trout with the recombinant protein. J. Gen. Virol. 74 (1993) 623-630) is made as follows: a unique BamHI site is introduced by PCR amplification of VHSV G sequence and primers V-HSVGSmaI (5′-gatcgacccgggagatctaagtcatcagaccgtctgacttctggagaactgc-3′) and VHSVGBamHI (5′-tctggtggatccgcagatcactcaacgacctccgg-3′). PCR conditions are 30 sec. at 96° C., 30 sec. 60° C. and 45 sec. at 72° C. for 30 cycles using Pwo polymerase. The resulting fragment is excised with BamHI and SmaI and cloned in frame in unique BamHI site of pLANTIGEN4 harbouring the synthetic gene for LTB. The resulting gene sequence under control of patatin promoter and nopaline synthase terminator sequence is named pLANTIGEN24 (pL24). A co-transformation of potato is performed with pL4and pL24 generating numerous pL(4+24) plants. The presence of pL4 and/or pL24 gene constructs is confirmed by PCR as described before. Plants that are positive for both gene constructs are allowed to form tubers. Tubers are harvested and analysed for GM1-binding complexes using GM1-ELISA. The presence of VHSV G protein in complexes is confirmed by incubation with monoclonal antibodies IP1H3, 3F1H10 and 3F1A2 (Lorenzen et al., 2000. Three monoclonal antibodies to the VHS virus glycoprotein: comparison of reactivity in relation to differences in immunoglobulin variable domain gene sequences. Fish & Shellfish immunology 10: 129-142). Chimeric complexes can further be characterized by Western blotting of tuber extracts run on SDS-PAGE under semi-native conditions and using the anti LTB5 mAb VD12 and 1P1H3, 3F1H10 and 3F1A2.

### Example 12

### Chimeric Complex of LTB and LTB-SVCV G

A BamHI/BglII fragment comprising the complete SVCV G gene of spring viremia of carp virus (Genbank accession nr. NC002803) and for making a genetic fusion with LTB, was amplified using oligonucleotides SVCVG1 (5′-tctggtctcgagatccccatatttgttccatc-3′) and SVCVG2 (5′- gatcgaggatccaagtcatcaaactaaagaccgcatttcg-3′). The resulting fragment was excised with BamHI and XhoI and cloned in the BamHI/XhoI site of pL4 coding for LTB; p thereby generating pLANTIGEN27 (pL27). The resulting gene placed under control of the tuber specific patatin promoter (pLANTIGEN27) was introduced in A.tumfaciens for transformation of potato. A co-transformation of pL4 and pL27 was performed and transgenic plants were evaluated for the presence of both gene constructs by PCR as described. Transgenic plants harbouring pL(4+27) gene constructs were selected and grown to maturity in the greenhouse. Tubers were analysed for accumulation of GM1-binding complexes by GM1-ELISA and for the presence of SVCV G protein using specific mAbs. The subunit com position of the protein complexes was visualized by Western blotting after semi-native SDS-PAGE as described.

### Example 13

### Chimeric Complex LTB and LTB-ClyIIA

Actinobacillus pleuropneumoniae serotype 9, reference strain CVI13261, is grown on heart infusion agar (Difco) containing 0.1% V-factor (NAD). High molecular weight DNA is isolated by proteinase K/SDS lysis, followed by phenol/chloroform extraction and precipitation of resulting genomic DNA. The ClyIIA gene from Actinobacillus pleuropneumoniae serotype 9 (GenBank-EMBL-accession nr. X61111) is cloned from genomic DNA isolated of serotype 9 strain by PCR using oligonucleotides Cyto11 (5′-gatccatggcaaaaatcactttgtcatc-3′) and Cyto14 (5′-atcggatccctattaagcggctctagctaattg-3′). Subsequently, a BamHI site is also introduced at the amino terminus of the ClyIIA gene by P-CR and the resulting fragment excised with BamHI was cloned in pET-wiLTB as described for expression in E.coli and generating pET-wiLTB-ClyIIA Chimeric complexes can be obtained by co-expression of pET-wiLTB and pET-wiLTB-ClyIIA upon induction with IPTG. Alternatively, inclusion bodies obtained upon overexpression of pET-wiLTB-ClyIIA in E.coli and purified recLTB are mixed and solubilized by 8M Urea and dialysed against Tris buffer as described to renature pentameric complexes.

### Example 14

### Multivalent Protein Complex Comprising LTB, LTB-H5 and LTB-N1

Synthetic genes for influenza A virus subtype H5N1 hemagglutinin, (Genbank accession nr. AF028709) and neuraminidase (AF028708; Claas et al. (1998) Human influenza A H5N1 virus related to a highly pathogenic avian influenza virus. Lancet 351 (9101), 472-477), both optimized for expression in plants are made. At the amino and carboxyl termini of H5 and N1 sequences, BamHI sites are introduced and genetic fusions are made by cloning the respective BamHI fragments comprising the synthetic H5 gene or N1 gene into the unique BamHI site of pLANTIGEN4 gene construct. The resulting genes coding for LTB-H5 and LTB-N1 fusion proteins, are cloned behind a patatin promoter as described in Example 1. A co-transformation of LTB, LTB-H5 and LTB-N1 is performed and potato plants are analysed for the presence of the respective genes by PCR as described before. Plants transgenic for all three expression cassettes can be isolated and further grown to maturity in the greenhouse. Tubers are analysed for the presence GM1-binding complexes by GM1-ELISA and Western blotting using VD12 and H5 and N1 specific antibodies. In another embodiment plants are generated harbouring either LTB-H5 or LTB-N1 and analysed for accumulation of respective complexes recLTB-H5 and recLTB-N1 can be purified from tubers and mixed with purified recLTB in a 1:1:3, 1:2:2, 1:3:1, 2:1:2, 2:2:1 or:3:1:1 respectively and solubilized and denatured using 8 M Urea and further treated as described in Example 9 to generate chimeric complexes.

Table 1

Comparison of means of molar expression data of homopentameric protein complexes produced in transgenic potato tubers harboring either one of the constructs pLANTIGEN4₁ 12, 133 and 15 with respective standard deviations and co-variance as derived from GM1 ELISA. Pairwise differences between constructs were analysed using ANOVA after log transformation of data to stabilise variance. Expression levels are expressed as mM. The last column shows the standard deviation (Sd).

| | Mean | |
|---|---|---|
| | Expression | |
| Construct | (mM) | Sd |
| pL4 | 102.00 | 70.69 |
| pL12 | 59.78 | 41.42 |
| pL15 | 25.17 | 14.31 |
| pL13 | 1.57 | 1.47 |

Table 2

Comparison of expression data of transgenic potato tubers harbouring chimeric protein complexes comprising pL4 as well as pL13 as derived from GM1 ELISA. Transgenic nature and presence of pL4 and/or pL13 gene constructs (second and third column) was by PCR of genomic DNA isolated of individual plants as described Example 4. The presence of chimeras was established by comparison of results of semi-native Western blot analysis using VD12 and V3 mAbs as described and screening for the presence of high molecular weight complexes as in FIG. 2 (fourth column). Expression data are expressed in micrograms of GM1-binding LTB5 moiety per gram fresh weight tuber as determined by GM1-ELISA (fifth column). For comparison, tubers comprising only the pL4 gene construct contained on average 10-15 μg/g FW LTB5, whereas pL13(17), the highest expressor for pL13 gene construct, had less than 1 μg/g FW tuber.

TABLE 2

| Plant | pL4 | pL13 | Chimeras | LTB5 (μg/g FW) |
|---|---|---|---|---|
| pL(4 + 13)8 | + | + | no | 0.5 |
| pL(4 + 13)15 | + | + | yes | 3.1 |
| pL(4 + 13)16 | + | + | yes | 7.8 |
| pL(4 + 13)19 | + | + | no | 0.4 |
| pL(4 + 13)39 | + | + | yes | 6.7 |
| pL(4 + 13)46 | + | + | yes | 20.4 |
| pL(4 + 13)60 | + | + | yes | 3.8 |
| pL(4 + 13)64 | + | + | yes | 5.8 |

Table 3

Comparison of expression data of transgenic potato tubers harbouring chimeric protein complexes composed out of pL4 and pL12 as derived from GM1 ELISA. Transgenic nature and presence of pL4 and/or pL12 gene constructs (second and third column) was by PCR of genomic DNA isolated of individual plants as described Example 5 The presence of chimeras was established by comparison of results of semi-native Western blot analysis using VD12 and 3C9 mAbs as described and screening for the presence of high molecular weight complexes as in FIG. 2. Expression data are expressed in micrograms of GM1 binding moiety per gram fresh weight tuber as determined by GM1-ELISA.

TABLE 3

| Plant | pL4 | pL12 | Chimeras | LTB5 (μg/g FW) |
|---|---|---|---|---|
| pL(4 + 12)19 | + | + | yes | 3.9 |
| pL(4 + 12)20 | + | + | yes | 3.6 |
| pL(4 + 12)23 | + | + | yes | 13.8 |
| pL(4 + 12)25 | + | + | no | 1.2 |
| pL(4 + 12)31 | + | + | yes | 12.6 |
| pL(4 + 12)32 | + | + | no | 0.7 |
| pL(4 + 12)41 | + | + | yes | 8.8 |
| pL(4 + 12)42 | + | + | no | 1.3 |
| pL(4 + 12)46 | + | + | yes | 2.0 |
| pL(4 + 12)51 | + | + | yes | 9.1 |
| pL(4 + 12)52 | + | + | yes | 10.8 |
| pL(4 + 12)57 | + | + | yes | 7.8 |
| pL(4 + 12)62 | + | + | yes | 2.4 |
| pL(4 + 12)65 | + | + | yes | 1.3 |

### REFERENCES

Arakawa, T., Chong, D. K. X., Langridge, W. H. R. (1998) Efficacy of a food plant-based oral cholera toxin B subunit vaccine. Nature biotechnology 16: 292-297.
Arakawa, T., Chong, D. K. X., Merrit, J. L., Langridge, W. H. R. (1997) Expression of cholera toxin B subunit oligomers in transgenic potato plants. Transgenic Research 6: 403-413.
Bakker, H., Bardor, M., Molthoff, J. W., Gomord, V., Elbers, I., Stevens, L. H., Jordi, W., Lommen, A., Faye, L., Lerouge, P., Bosch, D. (2001) Galactose-extended glycans of antibodies produced by transgenic plants. Proceedings of the National Academy of Sciences of the USA 98: 2899-2904.
Bowen, J. C., Nair, S. K., Reddy, R., Rouse, B. T. (1994) Cholera toxin acts as a potent adjuvant for the induction of cytotoxic T-lymphocyte responses with non-replicating antigens Immunology 81: 338-342.
Bradford, M. M. (1976) A rapid and sensitive method for the quantitation of microgram quantities of protein utilizing the principle of protein-dye binding. Analytical biochemistry 72: 248-254.
Dallas, W. S., Falkow, S. (1980) Amino acid sequence homology between cholera toxin and Escherichia coli heat-labile toxin Nature 288: 499-501.
Dalsgaard, K., Uttenthal, Å, Jones, T. D., Xu, F., Merryweather, A., Hamilton, W. D. O., Langeveld, J. P. M., Boshuizen, R. S., Kamstrup, S, Lomonosoff, G. P., Porta, C., Vela, C, Casal, J. I., Meloen, R. H., Rodgers, P. B. (199 7) Plant-derived vaccine protects animals against a viral disease. Nature Biotechnology 15: 248-252.
Dertzbaugh, M. T., Elson, C. (1993) Comparative effectiveness of the cholera toxin B subunit and alkaline phosphatase as carriers for oral vaccines. Infection and Immunity 61: 48-55.
Deen, N. D. Van Holten, C., Claassen, E., Boersma, W. J. A. (1993) Peptide-induced memory (IgG) response, cross-reactive with native proteins, requires covalent linkage of a specific B cell epitope with a T cell epitope. European Journal Immunology 23: 630-634.
Engelen, F. A. van, Molthoff, J. W., Conner, A. J., Nap, J.-P., Pereira, A., Stiekema, W. J. (1995) pBINPLUS: an improved plant transformation vector based on pBIN19. Transgenic Research 4: 288-290.
Florack, D. E. A., Dirkse, W. G., Visser, B., Heidekamp, F., Stiekema, W. J. (1994) Expression of biologically active hordothionins in tobacco. Effects of pre- and pro-sequences at the amino and carboxyl termini of the hordothionin precursor on mature protein expression and sorting. Plant molecular Biology 24: 83-96.
Hackett, C. J., Dietzschold, B., Gerhard, W, Ghrist, B., Knorr, R., Gillessen, D., Melchers, F. (1983) The influenza virus site recognized by a murine helper T-cell specific for H1 strains: localization to nine amino acid sequence in the hemagglutinin molecule. Journal of Experimental Medicine 158: 294-
Hackett, C. J., Hurwitz, J. L., Dietzschold, B., Gerhard, W. (1985) Synthetic decapeptide of influenza virus hemagglutinin elicits helper T cells with the same fine recognition specificities as occur in response to whole virus. Journal of immunology 135: 1391-1394.
Haq, T. A., Mason, H. S., Clements, J. D., Arntzen, C. J. (1995) Oral immunization with a recombinant antigen produced in transgenic plants. Science 268: 714-716.
Hein, M. B., Yeo, T.-C., Wang, F., Sturtevant, A. (1995) Expression of cholera toxin subunits in plants. Annals of the New York Academy of Sciences 792: 50-56.
Holmgren, J. (1973) Comparison of the tissue receptors for Vibrio cholera and Escherichia coli enterotoxins by means of gangliosides and natural cholera toxoid. Infection and Immunity 8: 851-859.
Holmgren, J., Lönnroth, I., Svennerholm, L. (1973) Tissue receptor for cholera enterotoxin: postulated structures from studies with GM1 ganglioside and related glycolipids. Infection and Immunity 8: 208-214.
Holmgren, J., Lönnroth, I., Mánsson, J., Svennerholm, L. (1975) Interaction of cholera toxin and membrane GM1 ganglioside of small intestine. Proc. Natl. Acad. Sci. USA 72: 2520-2524.
Hulst, M. M., Westra, D. F., Wensvoort, G., Moormann, R. J. M. (1993) Glycoprotein E1 of hog cholera virus expressed in insect cells protects swine from hog cholera. Journal of Virology 67: 5435-3542.
Jagusztyn-Krynicka, E. K., Clark-Curtiss, J. E., Curtiss III, R. (1993) Escherichia coli heat-labile toxin subunit B fusions with Streptococcus sobrinus antigens expressed by Salmonella typhimurium oral vaccine strains: importance of the linker for antigenicity and biological activities of the hybrid protein Infection and Immunity 61: 1004-1015.
Kapusta, J., Modelska, A., Figlerowicz, M., Pniewski, T., Letellier, M., Lisowa, O., Yusibov, V., Koprowski, H., Plucienniczak, A., Legocki, A. B. (1999) A plant-derived edible vaccine against hepatitis B virus. FASEB XX:1796-1799.
Langridge, W. H. R. (2000) Edible vaccines. Scientific American September, 48-53.
Lauterslager, T. G. M., Florack, D. E. A., Van der Wal, T. J., Molthoff, J. W., Langeveld, J. P. M., Bosch, D, Boersma, W. J. A., Hilgers, L. A. Th. (2001) Oral immunisation of naive and primed animals with transgenic potato tubers expressing LT-B. Vaccine 19: 2749-2755.
Lazo, G. R., Stein, P. A., Ludwig, R. A. (1991) A DNA transformation-competent Arabidopsis genomic library in Agrobacterium. Biotechnology (NY) 9: 963-967.
Leong, J., Vinal, A. C. Dallas, W. S. (1985) Nucleotide sequence comparison between heat-labile toxin B-subunit cistrons from Escherichia coli of human and porcine origin. Infection and Immunity 48: 73-77.
Mason, H. S., Arntzen, C. J. (1995) Transgenic plants as vaccine production systems TIBTECH 13: 388-392.
Mason, H. S., Ball, J. M., Shi, J.-J., Jiang, X., Estes, M. K., Arntzen, C. J. (1996) Expression of Norwalk virus capsid protein in transgenic tobacco and potato and its oral immunogenicity in mice Proceedings of the National Academy of Sciences of the USA 93: 5335-5340.
Mason, H. S., Haq, T. A., clements, J. D., Arntzen, C. J. (1998) Edible vaccine protects mice against Escherichia coli heat-labile enterotoxin (LT): potatoes expressing a synthetic LT-B gene. Vaccine 16: 133 6-1343.
Mason, H. S., Lam, D. M.-K., Arntzen, C. J. (1992) Expression of hepatitis B surface antigen in transgenic plants Proceedings of the National Academy of Sciences of the USA 89: 11745-11749.
Mor, T. S., Gómez-Lim, M. S., Palmer, K. E. (1998) Perspective: edible vaccines, a concept coming of age. Trends in Microbiology 6: 449-453.
Munro, S., Pelham, H. R. B. (1987). A C-terminal signal prevents secretion of luminal ER proteins. Cell 48: 899-907.
Richter, L., Kipp, P. B. (1999) Transgenic plants as edible vaccines. Current Topics in Microbiology and Immunology 240: 159-176.
Sala, F. et al. (2003). Vaccine antigen production in transgenic plants: strategies, gene constructs and perspectives Vaccine 21: 803-808.
Sanger, F., Nicklen, S., Coulson, A. R. (1977). DNA sequencing with the chain-terminating inhibitors. Proceedings of the National Academy of Sciences of the USA 74: 5463-5467.
Schouten, A., Roosien, J., Van Engelen, F. A., De Jong, G. A. M, Borst-Vrenssen, A. W. M., Zilverentant, J. F., Bosch, D. (1996) The C-terminal KDE1 sequence increases the expression level of a single-chain antibody designed to be targeted to both the cytosol and the secretory pathway in transgenic tobacco. Plant Molecular Biology 30: 781-793.
Spangler, B. D. (1992) Structure and function of cholera toxin and the related Escherichia coli heat-labile enterotoxin. Microbiological Reviews 56: 622-647.
Streatfield, S. J. Jilka, J. M., Hood, E. E., Turner, D. D. , Bailey, M. R., Mayor, J. M., Woodard, S. L., Beifuss, K. K., Horn, M. E., Delaney, D. E., Tizard, I. R., Howard, J. A. (2001) Plant-based vaccines: unique advantages. Vaccine 19: 2742-2748.
Tacket, C. O., Mason, H. S. (1999) A review of oral vaccination with transgenic vegetables Microbes and Infection 1: 777-783.
Tacket, C. O., Mason, H. S., Losonsky, G., Clements, J. D., Levine, M. M., Arntzen, C. J. (1998) Immunogenicity in humans of a recombinant bacterial antigen delivered in a transgenic potato. Nature Medicine 4: 607-609.
Thanavala, Y., Yang, Y.-F., Lyons, P., Mason, H. S., Arntzen, C. (1995) Immunogenicity of transgenic plant-derived hepatitis B surface antigen. Proceedings of the National Academy of Sciences of the USA 92: 335 8-3361.
Towbin, H., Staehelin, T., Gordon, J. (1979) Electrophoretic transfer of proteins from polyacrylamide gels to nitrocellulose sheets: procedures and some applications. Proceedings of the National Academy of Sciences of the USA 76: 4350-5354.

1. A protein complex comprising at least two, preferably identical, subunits wherein at least one subunit is unaltered and at least one subunit is fused to a first molecule of interest and wherein the protein complex is able to interact with a cell surface receptor via said subunits.

2. A protein complex according to claim 1, wherein said first molecule of interest can associate with, preferably via a covalent bond, a second molecule of interest to form a multimer of interest.

3. A protein complex according to claim 1, wherein said complex is essentially based on the heat labile enterotoxin (LT) of E. coli or on the cholera toxin (CT) of Vibrio cholerae, preferably the B subunits thereof.

4. A protein complex according to claim 1, wherein said complex comprises at least two subunits provided with a molecule of interest.

5. A protein complex according to claim 4, wherein said at least two subunits are provided with a different molecule of interest.

6. A protein complex according to claim 1, wherein said cell surface receptor is present on intestinal epithelial.

7. A protein complex according to claim 1, wherein at least one molecule of interest is an antigen.

8. A protein complex according to claim 7, wherein said antigen is selected from the group consisting of a bacterial antigen, a viral antigen, a protozoal antigen and a fungal antigen.

9. A protein complex according to claim 1, wherein at least one molecule of interest is an immunomodulatory protein, preferably a cytokine or a heat-shock protein.

10. A protein complex according to claim 1, wherein said complex comprises five B subunits of the heat labile enterotoxin (LT) of E. coli or the cholera toxin (CT) of Vibrio cholerae, wherein at least one subunit is unaltered.

11. A protein complex according to claim 1, wherein said cell surface receptor comprises a ganglioside molecule, preferably GM1, or a mimic thereof.

12. A method for producing a protein complex according to claim 1, comprising:
a) providing a host cell with a nucleotide sequence encoding an unaltered subunit and a nucleotide sequence encoding a molecule of interest, wherein at least one molecule of interest is fused to a subunit;
b) culturing said host cell thereby allowing expression of said nucleotide sequences and allowing for assembly of the protein complex;
c) isolating the complex; and
d) determining the binding of the complex to a cell surface receptor or to a molecule which mimics a cell surface receptor.

13. A method for producing a protein complex according to claim 1, comprising:
a) providing a first host cell with a nucleotide sequence encoding an unaltered subunit and a second host cell a nucleotide sequence encoding a molecule of interest, wherein at least one molecule of interest is fused to a subunit;
b) culturing said host cells thereby allowing expression of said nucleotide sequences;
c) isolating the proteins encoded by said nucleotides;
d) contacting the isolated protein under conditions allowing for assembly of the protein complex;
e) isolating the complex; and
f) determining the binding of the complex to a cell surface receptor or to a molecule which mimics a cell surface receptor.

14. A method according to claim 13, wherein said host cell is provided with said nucleotide sequences using transformation, co-transformation, crossing, re-transformation or transient transfection.

15. A cell comprising the protein complex according to claim 1.

16. A cell according to claim 15, wherein said cell is a plant cell.

17. A cell according to claim 15, wherein said cell is an edible cell.

18. A composition comprising a protein complex according to claim 1.

19. A vaccine comprising a protein complex according to claim 1 and a pharmaceutically acceptable carrier.

20. A pharmaceutical composition comprising an effective amount of a vaccine according to claim 19.

21. Use of a protein complex according to claim 1 as a mucosal carrier molecule.

22. A method for modulating an immune response of a subject comprising administering to the subject at least one dose of an effective amount of a protein complex according to claim 1, wherein the molecule of interest is an antigen.

23. A method for mucosal immunisation comprising the administration of a vaccine according to claim 19 to a subject.

24. A composition comprising a cell according to claim 15.

25. A vaccine comprising a cell according to claim 15 and a pharmaceutically acceptable carrier.
